SEQUENCE LISTING (13 sequences)

SEQ ID NO: 1 - protein, 117 aa, Homo sapiens
CLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAA

SEQ ID NO: 2 - DNA, 351 bp, Homo sapiens
tgcctgcccctgccaccctccctgccagtctgcggccacctgggcatctcacgcttctggctgcccaaccacctccaccacgagagcggcgagcaggtgcgggccggggcacgggcgtgggggggcctgctgcagacgcactgccatcccttcctcgcctggttcttctgcctgctgctggtccccccatgcggcagcgtcccgccgcccgccccgccaccctgctgccagttctgcgaggccctgcaggatgcgtgttggagccgcctgggcgggggccggctgcccgtcgcctgtgcctcgctcccgacccaggaggatgggtactgtgtgctcattgggccggctgca

SEQ ID NO: 3 - protein, 497 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARY

SEQ ID NO: 4 - DNA, 1491 bp, Homo sapiens
atggctccctacccctgtggctgccacatcctgctgctgctcttctgctgcctggcggctgcccgggccaacctgctgaacctgaactggctttggttcaataatgaggacaccagccacgcagctaccacgatccctgagccccaggggcccctgcctgtgcagcccacagcagataccaccacacacgtgaccccccggaatggttccacagagccagcgacagcccctggcagccctgagccaccctcagagctgctggaagatggccaggacacccccacttctgccgagagcccggacgcgccagaggagaacattgccggtgtcggagccgagatcctgaacgtggccaaaggcatccggagcttcgtccagctgtggaatgacactgtccccactgagagcttggccagggcggaaaccctggtcctggagactcctgtgggcccccttgccctcgctgggccttccagcaccccccaggagaatgggaccactctctggcccagccgtggcattcctagctctccgggcgcccacacaaccgaggctggcaccttgcctgcacccaccccatcgcctccctccctgggcaggccctgggcaccactcacggggccctcagtgccaccaccatcttcaggtagagcttctctctcctccttgctgggcggggcccctccctggggaagcctgcaggacccagacagccaaggactctcgcccgccgcagccgctcccagccagcagctccaacgccctgacgtccgcctgcgcacgccacttctgcaccccctggtgatgggctccctgggcaagcacgcggccccctccgccttctcctctgggctcccgggcgcactgtctcaggtcgcagtcaccactttaaccagggacagcggtgcttgggtctcccacgtggctaactctgtggggccgggtcttgctaataactctgccctgctcggggctgaccccgaggcccccgccggtcgctgcctgcccctgccaccctccctgccagtctgcggccacctgggcatctcacgcttctggctgcccaaccacctccaccacgagagcggcgagcaggtgcgggccggggcacgggcgtgggggggcctgctgcagacgcactgccatcccttcctcgcctggttcttctgcctgctgctggtccccccatgcggcagcgtcccgccgcccgccccgccaccctgctgccagttctgcgaggccctgcaggatgcgtgttggagccgcctgggcgggggccggctgcccgtcgcctgtgcctcgctcccgacccaggaggatgggtactgtgtgctcattgggccggctgcagagcgcatcagcgaggaggtggggctgctgcagctccttggggaccccccgccccagcaggtcacccagacggatgaccccgacgtcgggctggcctacgtctttgggccagatgccaacagtggccaagtggcccggtac

SEQ ID NO: 5 - protein, 235 aa, Homo sapiens
GRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAA

SEQ ID NO: 6 - protein, 23 aa, Homo sapiens
MAPYPCGCHILLLLFCCLAAARA

SEQ ID NO: 7 - DNA, 69 bp, Homo sapiens
atggctccctacccctgtggctgccacatcctgctgctgctcttctgctgcctggcggctgcccgggcc

SEQ ID NO: 8 - protein, 15 aa, Homo sapiens
AWGGLLQTHCHPFLA

SEQ ID NO: 9 - protein, 15 aa, Mus musculus
AWGRFLHTNCHPFLA

SEQ ID NO: 10 - DNA, 21 bp, artificial
  primer
gcttctctctcctccttgctg

SEQ ID NO: 11 - DNA, 20 bp, artificial
  primer
gagagtccttggctgtctgg

SEQ ID NO: 12 - DNA, 1629 bp, Homo sapiens
atggctccctacccctgtggctgccacatcctgctgctgctcttctgctgcctggcggctgcccgggccaacctgctgaacctgaactggctttggttcaataatgaggacaccagccacgcagctaccacgatccctgagccccaggggcccctgcctgtgcagcccacagcagataccaccacacacgtgaccccccggaatggttccacagagccagcgacagcccctggcagccctgagccaccctcagagctgctggaagatggccaggacacccccacttctgccgagagcccggacgcgccagaggagaacattgccggtgtcggagccgagatcctgaacgtggccaaaggcatccggagcttcgtccagctgtggaatgacactgtccccactgagagcttggccagggcggaaaccctggtcctggagactcctgtgggcccccttgccctcgctgggccttccagcaccccccaggagaatgggaccactctctggcccagccgtggcattcctagctctccgggcgcccacacaaccgaggctggcaccttgcctgcacccaccccatcgcctccctccctgggcaggccctgggcaccactcacggggccctcagtgccaccaccatcttcaggtagagcttctctctcctccttgctgggcggggcccctccctggggaagcctgcaggacccagacagccaaggactctcgcccgccgcagccgctcccagccagcagctccaacgccctgacgtccgcctgcgcacgccacttctgcaccccctggtgatgggctccctgggcaagcacgcggccccctccgccttctcctctgggctcccgggcgcactgtctcaggtcgcagtcaccactttaaccagggacagcggtgcttgggtctcccacgtggctaactctgtggggccgggtcttgctaataactctgccctgctcggggctgaccccgaggcccccgccggtcgctgcctgcccctgccaccctccctgccagtctgcggccacctgggcatctcacgcttctggctgcccaaccacctccaccacgagagcggcgagcaggtgcgggccggggcacgggcgtgggggggcctgctgcagacgcactgccatcccttcctcgcctggttcttctgcctgctgctggtccccccatgcggcagcgtcccgccgcccgccccgccaccctgctgccagttctgcgaggccctgcaggatgcgtgttggagccgcctgggcgggggccggctgcccgtcgcctgtgcctcgctcccgacccaggaggatgggtactgtgtgctcattgggccggctgcagagcgcatcagcgaggaggtggggctgctgcagctccttggggaccccccgccccagcaggtcacccagacggatgaccccgacgtcgggctggcctacgtctttgggccagatgccaacagtggccaagtggcccggtacaagggcaattctgcagatatccagcacagtggcggccgctcgagtctagagggcccgcggttcgaaggtaagcctatccctaaccctctcctcggtctcgattctacgcgtaccggtcatcatcaccatcaccattga

SEQ ID NO: 13 - DNA, 975 bp, homo sapiens
atggagacagacacactcctgctatgggtactgctgctctgggttccaggttccactggtgacgcggcccagccggccaggcgcgcgcgccgtacgtacgaagcttggtaccgagctcggatccactccagtgtggtggaattcgcccttggtagagcttctctctcctccttgctgggcggggcccctccctggggaagcctgcaggacccagacagccaaggactctcgcccgccgcagccgctcccagccagcagctccaacgccctgacgtccgcctgcgcacgccacttctgcaccccctggtgatgggctccctgggcaagcacgcggccccctccgccttctcctctgggctcccgggcgcactgtctcaggtcgcagtcaccactttaaccagggacagcggtgcttgggtctcccacgtggctaactctgtggggccgggtcttgctaataactctgccctgctcggggctgaccccgaggcccccgccggtcgctgcctgcccctgccaccctccctgccagtctgcggccacctgggcatctcacgcttctggctgcccaaccacctccaccacgagagcggcgagcaggtgcgggccggggcacgggcgtgggggggcctgctgcagacgcactgccaccccttcctcgcctggttcttctgcctgctgctggtccccccatgcggcagcgtcccgccgcccgccccgccaccctgctgccagttctgcgaggccctgcaggatgcgtgttggagccgcctgggcgggggccggctgcccgtcgcctgtgcctcgctcccgacccaggaggatgggtactgtgtgctcattgggccggctgcagaagggcgaattctgcagatatccagcacagtggcggccgctcgaggagggcccgaacaaaaactcatctcagaagaggatctgaatagcgccgtcgaccatcatcatcatcatcattga

(19) United States
(12) Patent Application Publication
(10) Pub. No.: US 2010/0316616 A1
(43) Pub. Date: Dec. 16, 2010

(54) Use of FZC18-Containing Collagen 18 Polypeptides for the Treatment, Diagnosis and Outcome Prediction of Diseases

(75) Inventors: Bruno Clement, Rennes (FR); Orlando Musso, Laille (FR); Delphine Quelard, Seattle, WA (US); Julie Leseur, Rennes (FR); Ismail Hendaoui, Rennes (FR); Elise Lavergne, Rennes (FR)

Correspondence Address: MCANDREWS HELD & MALLOY, LTD, 500 WEST MADISON STREET, SUITE 3400, CHICAGO, IL, 60661, US

(73) Assignee: INSERM (Institut National de la Sante et de la Recherche Medicale), Paris (FR)

(21) Appl. No.: 12/808,580

(22) PCT Filed: Dec. 17, 2008
(86) PCT No.: PCT/EP08/67779

§ 371 Date: Jun. 16, 2010

(30) Foreign Application Priority Data
Dec. 17, 2007 (EP) 07301681.8

(51) Int. Cl. A61K 35/23; C07K 7/08; C07K 14/47; C07H 21/04; C12N 5/10; A61K 38/17; A61P 35/00; A61K 31/711; G01N 33/68

(52) U.S. Cl. 424/93.21; 530/327; 530/326; 530/325; 530/324; 530/350; 536/23.5; 435/369; 514/19.3; 514/19.4; 514/19.5; 514/44 R; 436/86

(57) ABSTRACT

The present invention relates to a polypeptide comprising at least 13 consecutive amino acids selected from the amino acid sequence as set forth in SEQ ID NO: 1 or a variant thereof comprising at least 70% identity over said 13 consecutive amino acids, wherein said polypeptide or variant thereof interacts with Wnt3a and can be used for the treatment of diseases associated with increased beta-catenin pathway activity. The present invention also relates to a method for detecting the presence of a disease associated with fibrogenesis and to a method for assessing the severity and/or predicting the outcome of cancer.

### FIELD OF THE INVENTION

The present invention relates to the use of a polypeptide or a nucleic acid for the treatment of diseases associated with an activated Wnt/beta-catenin signaling pathway such as cancer, for the diagnosis of diseases associated with fibrogenesis and for predicting the outcome of cancer.

### BACKGROUND OF THE INVENTION

Wnt proteins are a family of cysteine-rich, secretory glycoproteins of approximately 40 kDa, and are known to be involved in various cell developmental processes including cell polarity (Moon et al., 2002). In humans, 19 wnt proteins have been reported, and 10 frizzled proteins as Wnt receptors and 2 coreceptors (LPR 5 and 6) are known (He et al., 2004).

Canonical Wnt signaling induces stabilization and accumulation of cytoplasmic beta-catenin through the regulation of a protein kinase complex and translocation of beta-catenin into the nucleus where it acts as a transcriptional activator. This transcriptional activity is reported to be caused by transcription factors in the group of the LEF/TCF (Moon et al., 2002; Reya and Clevers, 2005; Wodarz and Nusse, 1998). In the absence of Wnt, beta-catenin is recruited into a destruction complex, phosphorylated at conserved N-terminal residues by GSK3β, and thus tagged for proteasomal degradation (Clevers, 2006). In the nucleus, target genes of the pathway are repressed by co-repressor binding to T-cell factor (TCF) transcription factors. Wnt binding to frizzled (FZ) receptors blocks beta-catenin phosphorylation, which becomes resistant to protcasomal degradation. Consequently, beta-catenin accumulates both in the cytoplasm and the nucleus, displacing co-repressors from TCF and activating transcription of target genes that regulate the balance between proliferation and apoptosis, differentiation and metabolism, including cyclin D1, c-myc (Clevers, 2006) and glutamine synthetase (GS) (Benhamouche et al., 2006).

Mutations or epigenetic silencing of components of the Wnt/beta-catenin signal transduction pathway are closely associated with increased cell growth (through increased proliferation or decreased cell death) and particularly, is also believed to be related to oncogenesis, such as colorectal cancer. For example, Wnt/beta-catenin signaling can be activated in human cancers by oncogenic mutations or by epigenetic silencing of pathway components. Respectively, 85% and 10% of sporadic colorectal cancers (CRCs) have APC and beta-catenin mutations (Clevers, 2006). In 30-40% of human hepatocellular carcinomas (HCCs), activation of the pathway results from beta-catenin and axin mutations (Laurent-Puig and Zucman-Rossi, 2006) or epigenetic silencing of Secreted Frizzled-Related Protein 1 (SFRP1) (Shih et al., 2006).

Indeed, FZ-Wnt interaction can be inhibited by Secreted Frizzled-Related Proteins (SFRPs) which work as decoy Wnt receptors quenching Wnts at the cell surface or, alternatively, directly interacting with FZ receptors. Five SFRPs arc known (SFRP1, SFRP2, SFRP3, SFRP4 and SFRP5). Other extracellular inhibitors of the Wnt/beta-catenin pathway activity are DKKs, which block interaction of Wnts with the LRP co-receptors.

Therefore, methods for treating cancer by using an agent that can inhibit the binding of the Wnt proteins to their frizzled receptor have been suggested in the art. For example, document WO98/54325 discloses the use of a secreted protein that contains a region homologous to the ligand binding domain of a cytokine receptor. This protein, called Frizzled-related protein (FRP), antagonizes the signaling of the Wnt family of cytokines and so can be used for the design of new cancer therapies.

Very little is known about the specificity of Wnt family members for various FZ receptors. Recent data suggest that one Wnt can activate at least two different pathways, possibly through activation of different receptors (Tao et al., 2005), indicating that the binding specificities of Wnts and, therefore, the resulting biological effects depend on receptor context. Thus, in the model proposed in the art, Wnt signaling is not intrinsically regulated by the Wnt proteins themselves, but by the availability of receptors (Mikels and Nusse, 2006). Receptor availability is not only regulated by receptor expression at the cell surface, but also by SFRPs acting as decoy receptors. However, data are scarce concerning the specificity of SFRP family members for various Wnts. Recent studies using Wnt3a, which is well known in the art as a prototypical Wnt, indicate that several SFRPs can bind to Wnt3a in the nanomolar range (Galli et al., 2006; Wawrzak et al., 2007), but the specificity or binding affinity of SFRPs for other Wnt family members await further studies.

Collagen 18 (C18) (Muragaki et al., 1995; Rehn et al., 1994), the parent molecule of endostatin (O'Reilly et al.,)997), is expressed as three distinct variants by two separate promoters and alternative splicing of one of the transcripts (Muragaki et al.. 1995; Rehn et al., 1996). Promoter #1 generates variant #1 (V1), which is a ubiquitous structural basement membrane component. Alternative splicing of transcripts from promoter #2 generates variants #2 (V2) (Elamaa et al., 2003: Lietard et al., 2000) and #3 (V3) (Elamaa et al., 2003), which arc secreted under the control of both liver-specific and ubiquitous transcription factors. The V3 of C18 carries a 235-aa stretch with 10 conserved cysteines, bearing sequence and structural identities with the cysteine-rich domain (CRD) of the extracellular domain of the frizzled (FZ) receptors and the secreted frizzled-related proteins (SFRPs) (Xu and Nusse, 1998). The inventors named this module FZC18.

However, it was never planned to use polypeptides comprising sequences of the amino-terminus of the variant #3 of collagen 18 and more specifically of the FZC18 module for suppressing tumors.

A number of diseases associated with fibrogenesis are associated with collagen gene expression. In fibrotic conditions, such as liver cirrhosis, expression of collagen genes is increased as a result of injury to the liver and the resultant cooperation of injured hepatocytes with nonparenchymal cells of the liver. The excessive accumulation of collagen resulting from this injury leads to the impairment of normal functioning of the liver (Kivirikko, 1993).

In liver diseases such as hepatocellular carcinoma, collagen breakdown and deposition occurs as a result of the cooperation of neoplastic hepatocytes with nonparenchymal cells of the liver. In such disease, well-differenciated tumor cells proliferate along the preexisting matrix scaffold, preserving the trabecular tissue architecture. As the disease progresses, less differenciated cells appear and hepatocellular carcinoma increases in size. Subsequently, the once well-differenciated trabecular pattern is lost as angiogenesis and remodeling of the extracellular matrix occur.

Document WO 98/56399 describes methods directed to detecting or monitoring pathological liver conditions such as cirrhosis and hepatocellular carcinoma by determining the levels of collagen 18 in serum.

However, it was never planned to measure the expression of the variant 3 of collagen 18 in a biological sample obtained from a patient in order to detect or to assess the severity or to predict the outcome of a disease in a patient.

### SUMMARY OF THE INVENTION

The invention relates to a polypeptide comprising at least 13 consecutive amino acids selected from the amino acid sequence as set forth in SEQ ID NO: 1 or a variant thereof comprising at least 70% identity over said 13 consecutive amino acids, wherein said polypeptide or variant thereof binds to Wnt3a and is for use in therapy.

Another object of the invention relates to a nucleic acid comprising a nucleic acid sequence encoding a polypeptide or variant thereof according to the invention in frame with a nucleic acid sequence encoding a signal peptide, wherein said nucleic acid sequence encoding a signal peptide is upstream from said nucleic acid sequence encoding a polypeptide or variant thereof according to the invention.

The invention also relates to a polypeptide or variant thereof or a nucleic acid according to the invention for the treatment of a disease associated with increased Wnt/beta-catenin pathway activity.

Another object of the invention is a method for diagnosing a disease associated with fibrogenesis in a subject, wherein the expression of the variant 3 of collagen 18 is measured in a biological sample obtained from said subject.

Another object of the invention is a method for assessing the severity and/or predicting the outcome of a disease selected from the group consisting of colorectal cancers, hepatocellular carcinomas, childhood hepatoblastomas, melanoma, multiple myeloma, lymphoproliferative malignant diseases, breast cancers, desmoids tumors, gastric cancers, Wilms kidney tumors, medulloblastomas, ovarian endometrioid carcinomas, endometrial carcinomas, pancreatic carcinomas, prostate and thyroid carcinomas, wherein the expression of the variant 3 of collagen 18 is measured in a biological sample obtained from said subject.

### DETAILED DESCRIPTION OF THE INVENTION

### Definitions

The term C18 has its general meaning in the art refers to the Collagen 18 as described uragaki et al., 1995; Rehn and Pihlajaniemi, 1994). An exemplary of human C18 and its amino terminal end variants (variant 1; variant 2 and variant 3) is provided in GenBank database under accession number AH013565.

The term V3 denotes the full-length variant 3 of the collagen 18, containing the exon 3 sequence subject to alternative splicing. Indeed, V3 differs from the other two variants of collagen 18 in that it contains the exon 3. The exon 3 of V3 of C18 carries a frizzled module, homologous to the extracellular cystein-rich domain (CRD) of the frizzled receptors. An exemplary V3 of C18 containing the exon 3 sequence subject to alternative splicing is provided among the sequences under accession number AY484968. The sequence encoded by exon 3 of C18 is a 235 amino acid module, within the noncollagenous aminoterminus of V3 of C18 (Elamaa et al., 2003) that the inventors termed FZC18 module (SEQ ID NO:5). Only variant 3 contains the FZC18 module.

The term CRD of V3 denotes the 117 amino acid-long cystein-rich domain of variant 3 of collagen 18 which is found within the FZC18 module. The CRD of V3 is represented by the amino acid sequence as set forth in SEQ ID NO:1 and by the nucleotide sequence at set forth in SEQ ID NO:2.

The term V3Nter denotes an amino-terminal fragment of the variant V3 of collagen 18. It was identified by the inventors in human liver tissues as a protcolytically processed polypeptide derived from the amino terminus of variant 3 of C18. V3Nter is composed of 23 amino acids from the natural signal peptide of V3 of collagen 18+192 amino acids corresponding to the DUF-959 module of collagen 18 (for domain of unknown function 959)+235 amino acids corresponding to the whole FZC18 module+47 amino acids corresponding to C-terminal region of the amino terminal non-collagenous domain of C18 common to all variants. An exemplary human native V3Nter amino acid sequence is shown in SEQ ID NO:3. An exemplary human native nucleic acid sequence encoding V3Nter is shown in SEQ ID NO:4.

The term “signal peptide” is well known in the art. It refers to a peptide sequence which is present at the N-terminus of polypeptides which are synthesized by ribosomes associated with the endoplasmic reticulum. The signal peptide enables the export of the synthesized polypeptide from the cell onto the cell surface or into the extracellular medium. Exemplary sequences of signal peptides are the natural signal peptide of V3 of collagen 18: (SEQ ID NO:6, amino acid sequence and SEQ ID NO: 7, nucleotide sequence) and the signal peptide of the immunoglobulin IgK. A number of prediction algorithms are available and well known to the person skilled in the art for determining whether a given peptide acts as a signal peptide.

The skilled person can readily design, from the general knowledge of the genetic code and using conventional techniques of molecular biology, a nucleic acid sequence encoding a given amino acid sequence.

As used herein, the term “in frame” has its general meaning in the art and refers to the open reading frame encoded by a nucleic acid sequence.

The Wnt/beta-catenin signaling pathway comprises soluble Wnt ligands and their cognate cell surface frizzled (FZ) receptors, and the downstream intracellular signaling cascade. Wnt/beta-catenin pathway activity is the result of cell surface and intracellular interplays, the latter involving the beta-catenin phosphorylation complex (GSK3b, APC, e(c), resulting in the physiological responses of target cells that result from the exposure of cells to the extracellular Wnt ligands or the pathological responses resulting from oncogenic mutations or epigenetic silencing of pathway components. This pathway is well known for its role in embryogenesis and cancer, but is also involved in normal physiological processes in adult animals such as fibrogenesis.

As used herein, the expression “which binds to Wnt3a” refers to the ability of a given polypeptide to physically bind to the Wnt3a protein in vitro. Tests for assessing whether a given polypeptide binds to Wnt3a are known to the skilled person. Such tests can include, but arc not limited to, affinity chromatography techniques such as GST-pulldown, phage display, co-immunoprecipitation, competition assays, ELISA techniques, Surface Plasmon Resonance (SPR), etc.

One example of such a test is SPR, which can be carried out as follows:

SPR is performed using nitrilotriacetic (NTA) sensor chips on a BIAcore system (GE Healthcare, France), Ni⁺⁺ being omitted in the reference flow cell. The polypeptides to be tested are selected from the amino acid sequence as set forth in SEQ ID NO: 1, carrying a His-tag. They are solubilised in PBS and captured at low surface density. Binding and washings are performed in neutral buffer using a series of concentrations of Wnt3a protein as a prototype Wnt protein, but other Wnts could be tested. Untagged Wnt3a protein is commercially available (R&D Systems, Lille, France). It is produced by stably transfected mouse fibroblasts (L cells) and purified using protocols known in the art (http://www.stanford.edu/·rnusse/wntwindow.html). The unloaded reference flow cell (no binding of Wnt) is automatically subtracted from each sensogram. The sensograms are normalized, expressed as relative units and analyzed with commercially distributed software (GE Healthcare) using a global fitting procedure and kinetics models.

Another example of a test for assessing whether a given polypeptide binds to Wnt3a is the competition assay, wherein the given peptide competes with V3Nter for binding to Wnt3a carried out as follows:

Mouse His-tagged Wnt3a and mouse V3Nter cDNAs are cotransfected in HEK 293-EBNA cells. Cells are incubated in the presence (+) or absence (−) or the polypeptide to be tested. Cells arc lysed and the cell lysates are incubated with sheep-anti-mouse-IgG-coated Dynabeads M-280 (Dynal) conjugated with anti-His. After washing in RIPA buffer, complexes are eluted in denaturing sample buffer, resolved by 10% PAGE-SDS and immunoblotted with anti-His antibody (to detect Wnt3a) or with anti-DU-959 (to detect V3Nter). If the polypeptide to be tested binds to Wnt3a, the amount of V3Nter protein recovered (detected by the anti-DUF-959 antibody) is lower in the +) sample than in the (−) sample.

Typically, a polypeptide according to the invention, which binds to Wnt3a, inhibits the Wnt/beta-catenin signaling pathway. As used herein, the expression “which inhibits the Wnt/beta-catenin signaling pathway” refers to a compound which reduces the activation of the Wnt/beta-catenin signaling pathway, as measured by the levels of downstream messengers. One assay which can be used for determining whether a given compound inhibits the Wnt/beta-catenin signaling pathway is the quantification of beta-catenin—T-cell factor (TCF)-regulated transcription (CRT) from a TCF/LEF responsive reporter in various cell lines such as the CRC cell line HCT116 (Suzuki et al., 2004). Alternatively, the levels of total beta-catenin, non phosphorylated beta-catenin, c-myc and cyclin D1 can be measured in HCT116 cells. Alternatively, the activity of the cyclin D1 promoter can be assessed by reporter gene assays using the cyclin D1 promoter upstream of luciferase cDNA, as described (Lavoie et al. 1996).

### Polypeptides and Nucleic Acids and Uses Thereof

A first object of the invention relates to polypeptides comprising at least 13 consecutive amino acids selected from the amino acid sequence as set forth in SEQ ID NO: 1 or a variant thereof comprising at least 70% identity over said 13 consecutive amino acids, wherein said polypeptide or variant thereof binds to Wnt3a and is for use in therapy.

In a preferred embodiment, said variant comprises at least 75% identity over said 13 amino acids, even more preferably at least 80%, at least 85%, at least 90%, at least 95%, at least 97%.

In a preferred embodiment, the polypeptides of the invention comprise at least 14 amino acids, preferably at least 15 amino acids, selected from the amino acid sequence as set forth in SEQ ID NO: 1. In preferred embodiments, the variants of the invention comprise a amino acid sequence comprising at least 70% identity, preferably at least 75%, 80%, 85%, 90%, 95% or 97% identity over said 14, preferably 15 amino acids selected from the amino acid sequence as set forth in SEQ ID NO:1.

In one embodiment, the polypeptide of the invention comprises the amino acid sequence as set forth in SEQ ID NO:1, or a variant thereof comprising at least 80%, preferably at least 85%, 90%, 95%, 96%_{,} 97%, 98%, 99%, 99.5% or 99.9% identity with SEQ ID NO:1.

In one embodiment, the polypeptide of the invention consists in the amino acid sequence as set forth in SEQ ID NO:1 or a variant thereof comprising at least 80%, preferably at least 85%, 90%, 95%, 96%, 97%, 98%, 99%, 99.5% or 99.9% identity with SEQ ID NO:1.

Typically, the polypeptides or variants thereof of the invention comprise at most 600 amino acids. In a preferred embodiment, the polypeptides or variants thereof of the invention comprise at most 500 amino acids, preferably 400, 300, 200, even more preferably 100, 50, 40, 30, 25, 20, 15 amino acids.

Typically, the polypeptides or variants thereof of the invention are soluble.

The invention also relates to polypeptides comprising at least 13 consecutive amino acids selected from the amino acid sequence as set forth in SEQ ID NO: 5 or a variant thereof comprising at least 70% identity over said 13 consecutive amino acids, wherein said polypeptide or variant thereof binds to Wnt3a and is for use in therapy.

In one embodiment, the polypeptide of the invention comprises the amino acid sequence as set forth in SEQ ID NO:5, or a variant thereof comprising at least 80%, preferably at least 85%, 90%, 95%, 96%, 97%, 98%, 99%, 99.5% or 99.9% identity with SEQ ID NO:5.

In one embodiment, the polypeptide of the invention consists in the amino acid sequence as set forth in SEQ ID NO:5 or a variant thereof comprising at least 80%, preferably at least 85%, 90%, 95%, 96%, 97%, 98%, 99%, 99.5% or 99.9% identity with SEQ ID NO:5.

In one embodiment, the poly⁻peptides or variants thereof of the invention may comprise a tag. A tag is an epitope-containing sequence which can be useful for the purification of a the peptide or polypeptide it is attached to by a variety of techniques such as affinity chromatography, for the localization of said peptide or polypeptide within a cell or a tissue sample using immunolabeling techniques, the detection of said peptide or polypeptide by immunoblotting etc. Examples of tags commonly employed in the art are the GST (glutathion-S-transferase)-tag, the FLAG™-tag, the Strep-tag™, V5 tag, myc tag, His tag etc.

In onc embodiment, said variant may consist in the amino acid sequence as set forth in SEQ ID NO:3 and named V3Nter.

In one embodiment, the polypeptide may comprise the amino acid sequence set forth in SEQ ID NO: 8, consisting of AWGGLLQTHCHPFLA. In one embodiment, said polypeptide consists of the amino acid sequence as set forth in SEQ ID NO: 8. In one embodiment, said variant consists of the amino acid sequence as set forth in SEQ ID NO: 9 (mouse homologue of SEQ ID NO:8).

Typically said polypeptide or variant thereof may be used in combination with radiotherapy and hormone therapy.

Typically said polypeptide or variant thereof may also be used in combination with one or more agents selected from the group consisting of an anticancer agent, an antiemetic agent, an hematopoietic colony stimulating factor, an analgesic agent and an anxiolytic agent.

Polypeptides of the invention or variants thereof may be produced by any technique known per se in the art, such as, without limitation, any chemical, biological, genetic or enzymatic technique, either alone or in combination(s).

Knowing the amino acid sequence of the desired sequence, one skilled in the art can readily produce a relevant part of the said polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

Alternatively, the polypeptides of the invention or variants thereof can be synthesized by recombinant DNA techniques as is now well-known in the art. For example, these fragments can be obtained as DNA expression products after incorporation of DNA sequences encoding the desired polypeptide into expression vectors and introduction of such vectors into suitable eukaryotic or prokaryotic hosts that will express the desired polypeptide, from which they can be later isolated using well-known techniques.

Polypeptides of the invention or variants thereof can be used in an isolated (e.g., purified) form or contained in a vector, such as a membrane or lipid vesicle (e.g. a liposome).

Another object of the invention relates to a nucleic acid comprising a nucleic acid sequence encoding a polypeptide or variant thereof according to the invention in frame with a nucleic acid sequence encoding a signal peptide, wherein said nucleic acid sequence encoding a signal peptide is upstream from said nucleic acid sequence encoding the polypeptide or variant thereof according to the invention.

In one embodiment, said nucleic acid may comprise the nucleic acid sequence encoding V3Nter as set forth in SEQ ID NO: 4.

Nucleic acids of the invention may be produced by any technique known per se in the art, such as, without limitation, any chemical, biological, genetic or enzymatic technique, either alone or in combination(s).

A further object of the invention relates to the use of a vector comprising a nucleic acid construct of the invention for the manufacture of a medicament intended for the treatment of diseases associated with an increased Wnt/beta-catenin pathway activity, such as certain types of cancers.

Such vectors/nucleic acid constructs may comprise regulatory elements, such as a promoter, enhancer, terminator and the like, to cause or direct expression of said polypeptide upon administration to a subject. The vectors may further comprise one or several origins of replication and/or selectable markers. The promoter region may be homologous or heterologous with respect to the coding sequence, and provide for ubiquitous, constitutive, regulated and/or tissue specific expression, in any appropriate host cell, including for in vivo use.

Examples of plasmids include replicating plasmids comprising an origin of replication, or integrative plasmids, such as for instance pUC, pcDNA, pBR, and the like. Examples of viral vector include adenoviral, retroviral, herpesvirus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+cells, 293 cells, etc. Detailed protocols for producing such replication-defective recombinant viruses may be found for instance in WO95/14785, WO96/22378, U.S. Pat. No. 5,882,877, U.S. Pat. No. 6,013,516, U.S. Pat. No. 4,861,719, U.S. Pat. No. 5,278,056 and WO94/19478.

In one embodiment, the invention relates to a mammalian expression vector (for example pcDNA3.1 available from Invitrogen) containing the cDNA of V3Nter (SEQ ID NO: 4) in frame with a V5 tag, separated by a 47 amino acid spacer, as illustrated in the following Examples. The nucleotide sequence of the open-reading frame contained in said vector is as set forth in SEQ ID NO:12.

In one embodiment, the invention relates to a mammalian expression vector (for example pSecTag2 available from Invitrogen) encoding human FZC18 (SEQ ID NO:5) in frame with an IgK signal sequence and a C-terminal myc tag. The nucleotide sequence of the open-reading frame contained in said vector is as set forth in SEQ ID NO:13.

In one embodiment, the invention relates to mammalian cell lines stably expressing the polypeptide of the invention, such as stable HEK293T clones expressing the FZC18 polypeptide, or HCT116 colorectal cancer cell lines stably expressing the V3 Nter polypeptide. The invention relates to a HEK293T cell line stably transfected with mammalian expression vector comprising SEQ ID NO:13. The invention also relates to a HCT116 colorectal cancer cell line stably transfected with the mammalian expression vector as comprising SEQ ID NO:12. Stable mammalian cell lines can be obtained according to standard transfection protocols using vectors which comprise selectable markers, followed by selection by growth in an appropriate medium and can be used as sources for producing the purified polypeptide of the invention.

The invention also relates to the use of such cell lines for producing a polypeptide of the invention. The invention relates to a method for producing a polypeptide of the invention comprising the step of culturing a cell line as described in a culture medium suitable for the secretion of said polypeptide. In a preferred embodiment said culture medium does not contain any serum. The method includes the step of recovering said culture medium and optionally concentrating said medium and/or optionally purifying said polypeptide. Typically, when the polypeptide of the invention comprises a tag, said tag can be used for the purification step according to standard techniques in the art (immuno-precipitation, chromatography etc.).

### Therapeutic Methods

A further object of the invention relates to a method of treating diseases associated with increased Wnt/beta-catenin pathway activity comprising administering to a subject in need thereof a therapeutically effective amount of a polypeptide or variant thereof or a nucleic acid according to the invention.

In one embodiment, diseases associated with increased Wnt/beta-catenin pathway activity include certain cancers, i.e. malignant neoplastic diseases. In such diseases, the increased Wnt/beta-catenin pathway activity may be the result of, without limitation, increased Wnt ligand or FZ receptor function, decreased function of extracellular or intracellular pathway inhibitors or Wnt/beta-catenin pathway mutations, such as, but not limited to, beta-catenin, axin and APC. Such cancers may include, but arc not limited to, colorectal cancers, human hepatocellular carcinomas, childhood hepatoblastomas, melanoma, multiple myeloma, lymphoproliferative malignant diseases, breast cancers, desmoids tumors, gastric cancers. Wilms kidney tumors, medulloblastomas, ovarian endometrioid carcinomas, endometrial carcinomas, pancreatic carcinomas, prostate and thyroid carcinomas, etc.

In the context of the invention, the term “treating” or “treatment”, as used herein, means reversing, alleviating, inhibiting the progress of, or preventing the disorder or condition to which such term applies, or one or more symptoms of such a disorder or condition.

As used herein, the term “subject” denotes a mammal, such as a rodent, a feline, a canine, and a primate. Preferably a subject according to the invention is a human.

By a “therapeutically effective amount” of the polypeptide or variant thereof or the nucleic acid according to the invention is meant a sufficient amount of the ligand to treat said cancer, at a reasonable benefit/risk ratio applicable to any medical treatment. It will be understood, however, that the total daily usage of the polypeptide or the nucleic acid of the present invention will be decided by the attending physician within the scope of sound medical judgment. The specific therapeutically effective dose level for any particular subject will depend upon a variety of factors including the disorder being treated and the severity of the disorder, activity of the polypeptide or the nucleic acid employed; the specific composition employed, the age, body weight, general health, sex and diet of the patient, the duration of the treatment; drugs used in combination or coincidental with the specific polypeptide employed, and like factors well known in the medical arts. For example, it is well known within the skill of the art to start doses of the compound at levels lower than those required to achieve the desired therapeutic effect and to gradually increase the dosage until the desired effect is achieved.

The polypeptide or variant thereof or the nucleic acid according to the invention may be used in combination with any other therapeutic strategy for treating the disorders or conditions as above described (e.g. external radiotherapy, chemotherapy or cytokine therapy).

### Pharmaceutical Compositions

A further object of the invention relates to a pharmaceutical composition comprising an effective amount of a polypeptide or variant thereof or a nucleic acid according to the invention and pharmaceutically acceptable excipients or carriers.

Any therapeutic agent of the invention as above described may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of a polypeptide or a nucleic acid according to the invention may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, mixtures thereof and in oils. Under ordinary conditions of storage and use, these preparations contain a preservative to prevent the growth of microorganisms.

The polypeptide or variant thereof or the nucleic acid according to the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions arc prepared by incorporating the active compounds in the required amount in the appropriate solvent with various of the other ingredients enumerated above, as required, followed by filtered sterilization. Generally, dispersions arc prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations arc easily administered in a variety of dosage forms, such as the type of injectable solutions described above, but drug release capsules and the like can also be employed.

For parenteral administration in an aqueous solution, for example, the solution may be suitably buffered and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g. tablets or other solids for oral administration; time release capsules; and any other form currently used.

In one embodiment, the pharmaceutical composition may comprise cells stably expressing a polypeptide or variant thereof according to the invention. For example, the pharmaceutical composition may comprise HEK293T cells stably expressing the FZC18 polypeptide, or HCT116 cells stably expressing the V3Nter polypeptide. The cells may be encapsulated in alginate gel beads, as described in Desille et al., 2001, 2002 and Mahler et al., 2003. This vectorization approach enables a localized delivery of the polypeptide of the invention.

Compositions of the present invention may comprise a further therapeutic active agent. The present invention also relates to a kit comprising a polypeptide or a nucleic acid according to the invention and a further therapeutic active agent.

In one embodiment said therapeutic active agent is an anticancer agent. For example, said anticancer agents include but are not limited to fludarabine, gemcitabine, capecitabine, methotrexate, taxol, taxotere, mercaptopurine, thioguanine, hydroxyurca, cytarabine, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbiem, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, anthracyclines, MDR inhibitors and Ca²⁺ ATPase inhibitors.

Additional anticancer agents may be selected from but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anticancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, accthylleucine monocmanolamine, alizapride, azasetron, benzquinamide, bictanautine, bromopride, buclizine, clebopride, cyclizine, dunenhydrinate, diphenidol, dolasctron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinae, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

### Diagnostic Methods

A further object of the invention relates to a method for diagnosing a disease associated with fibrogenesis in a subject, wherein the expression of the variant 3 of collagen 18 is measured in a biological sample obtained from said subject

Typically, the invention relates to a method for diagnosing a disease associated with fibrogenesis in a subject, wherein the expression of proteolyzed forms of the variant 3 of collagen 18 is measured in a biological sample obtained from said subject.

As used herein, the expression “disease associated with fibrogenesis” refers to diseases in which extracellular matrix remodeling and fibrogenesis are enhanced. Indeed, proteolytic release of FZC18 and its precursors from full-length C18 was identified by the inventors in association with extracellular matrix remodelling.

The diseases associated with fibrogenesis include, but arc not limited to, hepatocellular carcinoma, renal or lung carcinomas, as well as inflammatory diseases wherein fibrogenesis is a hallmark tissue lesion, such as, but not limited to, interstitial kidney or pulmonary fibroses, viral or autoimmune hepatitis, liver fibrosis and cirrhosis of diverse aetiology.

Typically, the biological sample used for diagnosing a disease associated with fibrogenesis according to the method of the invention by assessing the extent of fibrogenesis can result from serum samples or from a biopsy, and more specifically from a liver biopsy, specimens of partial resection of a diseased part of an organ (e.g., partial hepatectomy, partial nephrectomy, and the like), whole organ explants performed in the case of orthotopic transplantations. Preferably, the measure of serum levels of the proteolyzed forms of C18 can constitute an attractive less invasive alternative than the analysis of tissue samples. In a preferred embodiment, the disease associated with fibrogenesis is selected from the group consisting of interstitial kidney fibroses, pulmonary fibroses, viral hepatitis, autoimmune hepatitis, liver fibrosis, liver cirrhosis, hepatocellular carcinoma, renal carcinoma and lung carcinoma.

In a preferred embodiment, the disease associated with fibrogenesis is a liver disease and said biological sample is a liver sample, such a biopsy sample.

In a preferred embodiment, the liver disease is liver fibrosis or liver cirrhosis or hepatocellular carcinoma.

Another object of the invention relates to a method for assessing the severity and/or predicting the outcome of a disease selected from the group consisting of colorectal cancers, hepatocellular carcinomas, childhood hepatoblastomas, melanoma, multiple myeloma, lymphoproliferative malignant diseases, breast cancers, desmoids tumors, gastric cancers, Wilms kidney tumors, medulloblastomas, ovarian endometrioid carcinomas, endometrial carcinomas, pancreatic carcinomas, prostate and thyroid carcinomas, wherein the expression of the variant 3 of collagen 18 is measured in a biological sample obtained from said subject.

According to this method, the assessment of the severity and/or prediction of the outcome is carried out after the diagnosis of the disease is first established using diagnostic methods conventionally used for such a disease and known to the skilled person in the art.

The biological sample may result from serum samples or a biopsy, and more specifically from a liver biopsy, specimens of partial resection of a diseased part of an organ (e.g., partial hepatectomy, partial nephrectomy, and the like) or whole organ explants performed in the case of orthotopic transplantations.

The expression of the variant 3 of collagen 18 can be measured at the level of the mRNA or at the level of the protein as follows:

Determination of the Expression Level of the Variant 3 of Collagen 18 by Quantifying mRNAs:

Total RNAs can be easily extracted from a biological sample. The biological sample may be treated prior to its use, e.g. in order to render nucleic acids or proteins available. Techniques of cell or protein lysis, concentration or dilution of nucleic acids, arc known by the skilled person.

Determination of the expression level of the variant 3 of collagen 18 can be performed by a variety of techniques. Generally, the expression level as determined is a relative expression level.

More preferably, the determination comprises contacting the sample with selective reagents such as probes, primers or ligands, and thereby detecting the presence, or measuring the amount, of or nucleic acids of interest originally in the sample. Contacting may be performed in any suitable device, such as a plate, microtiter dish, test tube, well, glass, column . . . . In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a 10 slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA may be then detected by hybridization (e.g., Northern blot analysis).

Alternatively, the extracted mRNA may be subjected to coupled reverse transcription and amplification, such as reverse transcription and amplification by polymerase chain reaction (RT-PCR), using specific oligonucleotide primers that enable amplification of a region in the nucleic acid defined by the SEQ ID NOs: 10 and 11. For example, forward primer: GCTTCTCTCTCCTCCTTGCTG, (SEQ ID NO: 10) and reverse primer: GAGAGTCCTTGGCTGTCTGG, (SEQ ID NO: 11) may be used. Quantitative or semi-quantitative RT-PCR is preferred. Real-time quantitative or semiquantitative RT-PCR is particularly advantageous. Extracted mRNA may be reverse transcribed and amplified, after which amplified sequences may be detected by hybridization with a suitable probe or by direct sequencing, or any other appropriate method known in the art.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarily or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

Probes typically comprise single-stranded nucleic acids of between 10 to 1000 nucleotides in length, for instance of between 10 and 800, more preferably of 15 between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

In a particular embodiment, the method of the invention the steps of providing total RNAs obtained from the biological sample of the patient, and subjecting the RNAs to amplification and hybridization to specific probes, more particularly by 25 means of a quantitative or semi-quantitative RT-PCR.

Total RNAs can be easily extracted from a biological sample. For instance, the biological sample may be treated prior to its use, e.g. in order to render nucleic acids available. Techniques of cell or protein lysis, concentration or dilution of nucleic acids, are known by the skilled person.

In another embodiment, the expression level may be determined by DNA microarray analysis. Such DNA microarray or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of 5 complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art [for a review see e.g. (Hoheisel, 2006)).

In this context, the invention further provides a DNA microarray comprising a solid support onto which nucleic acids that are specific for the nucleic acid of Genbank accession number AH013565 (i.e. mRNA or cDNA) are immobilized.

### Determination of the Expression Level of the Variant 3 of Collagen 18 by Quantifying Proteins:

Other methods exist for determining the expression level of the variant 3 of collagen 18.

Such methods comprise contacting a biological sample with a binding partner capable of selectively interacting with the variant 3 of collagen 18 present in the sample. The binding partner is generally an antibody that may be polyclonal or monoclonal, preferably monoclonal.

The presence of the variant 3 of collagen 18 can be detected using standard electrophoretic and immunodiagnostic techniques, including immunoassays such as competition, direct reaction, or sandwich type assays. Such assays include, but arc not limited to, Western blots; agglutination tests; enzyme-labeled and mediated immunoassays, such as ELISAs; biotin/avidin type assays: radioimmunoassays; immunoelectrophoresis; immunoprecipitation, immunocytochemistry, immunohistochemistry, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A biological sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

In one embodiment, the method of the invention further may comprise a step of comparing the concentration of the polypeptides comprising SEQ ID NO:1 or messenger RNA encoding said polypeptides with a predetermined value. Said comparison is indicative of outcome in patient.

In the following, the invention will be illustrated by means of the following examples as well as the figures.

### FIGURE LEGENDS

FIG. 1. V3Nter inhibits Wnt/beta-catenin signaling and downstream protein expression in cancer cells

(A) V3Nter and V2Nter expression vectors. Thick horizontal color lines indicate the antibodies used. Blue box, 47-aa stretch from Tsp-1C18.

(B) Dose-dependent changes in CRT in response to increasing amounts of transiently transfected cDNA vectors. Reporter gene assays using a beta-catenin-TCF reporter driven by wild-type (SUPER8XTOPFLASH, white bars) or a negative control with mutated TCF binding sites (SUPER8XFOPFLASH, black bars). Results arc means of three replicates from a representative experiment. Three independent experiments were performed. Error bars represent standard deviations.

(C) Immunoblot of HCT116 cells transiently transfected and probed with the indicated cDNA vectors (top) and antibodies (right). Hsc70 is a loading standard.

FIG. 2. V3FL does not inhibit Wnt/beta-catenin signaling.

(A) Schematic of V2FL and V3FL of C18 showing DUF-959, FZC18, Tsp-1C18 (thrombospondin-1) and endostatin (ES) modules. Thick horizontal lines indicate the antibodies used.

(B) Changes in CRT in response to increasing amounts of transiently transfected cDNA vectors. Reporter gene assays using a beta-catenin-TCF reporter driven by wild-type (SUPER8XTOPFLASH, white bars) or a negative control with mutated TCF binding sites (SUPER8XFOPFLASH, black bars). Results are means of three replicates from a representative experiment. Three independent experiments were performed. Error bars represent standard deviations.

(C) Reporter gene assays using a beta-catenin-TCF responsive reporter (SUPER8XTOPFLASH) in human HCC Huh-7 cells (wild-type be(a-catenin). Results are means of three replicates from a representative experiment. Three independent experiments were performed. Error bars represent standard deviations. P=(Student's “t” test) indicates statistical significance with respect to cells transfected with vector alone (VECTOR). NS, not significant.

(D) Reporter gene assays using cyclin D1 promoter reporter upstream luciferase cDNA in HCT116 and HepG2 cells. Results are means of three replicates from a representative experiment. Three independent experiments were performed. Error bars represent standard deviations. P=(Student's “1” test) indicates statistical significance with respect to cells transfected with vector alone (VECTOR). NS, not significant.

FIG. 3. V3Nter decreases colony formation and induces tumor cell death in cancer cells. HCT116 (A, C and D) and HepG2 (B, E) cells were transfected with cDNA vectors and selected for 14 d (A and B), 4 d (C and E) or 6-8 d (D) with G418.

(A and B) After hematoxylin staining, colonies (seen as dark spots) were digitized using a video camera and counted with Scion Image (NIH). Histograms show colony formation efficiencies relative to cells transfected with empty vector.

(C) Hoescht 33342-stained cells photographed at ×200 magnification. Arrows indicate apoptotic cells.

(D) Hoescht cell counts show mean±SD percent apoptotic cells out of triplicate 300-cell counts from 6-well plates (blindly at ×200 magnification) at each time point. SubG1 shows mean±SD percent apoptotic cells out of triplicate 1×10⁴ cells from 6-well plates assessed by flow cytometry at each time point.

(E) Representative FL2-H histograms of HepG2 cells show percent SubG1 population.

FIG. 4. FZC18 suppresses Wnt/beta-catenin signaling and clonogenesis in cancer cells.

(A) Reporter gene assays using a beta-catenin-TCF responsive reporter (SUPER8XTOPFLASH, white bars) and a negative control (SUPER8XFOPFLASH, black bars) in HCT116 cells. Dose-dependent decrease in CRT is detected in response to increasing amounts of transiently transfected FZC18 cDNA. Controls include SFRP1, SFRP5, V3Nter and V2Nter (250 ng cDNA).

(B) Immunoblot of HCT116 cells transiently transfected with increasing amounts of FZC18 cDNA. The blots were probed with the indicated antibodies (right). Hsc70 is a loading standard.

(C) Reporter gene assays using cyclin D1 promoter driving luciferase expression in transiently transfected HCT116 and HepG2 cells. Results are expressed relative to cells transfected with empty vector.

(D) Colony formation assay in HCT116 cells transfected with FZC18 or V2Nter. Histograms show colony formation efficiencies.

(E) TCF-reporter gene assays using SUPER8XTOPFLASH (white) and SUPER8XFOPFLASH (black). Huh-7 cells were transiently transfected with a constitutively active form of beta-catenin (Δ29-48) and with the indicated cDNAs. Bars represent mean±SD. Results are means of three replicates from a representative experiment. Three independent experiments were performed.

FIG. 5. V3Nter reduces in vitro tumor cell growth and modulates translocation of beta-catenin.

(A) Immunoperoxidase staining (brown) with anti-V5 epitope tag detects FZC18 in two clones of HCT116 CRC cells stably expressing V3Nter. Blue, hematoxylin counterstaining. [Original magnification ×200].

(B) Fluorescence microscopy of clone HCT116 V3Nter #1 incubated with mouse monoclonal anti-V5 epitope tag (to detect FZC18) and with rabbit polyclonal anti-beta-catenin antibodies followed by anti-mouse FITC (green) and anti-rabbit-TRITC (red) labeled IgGs. V3Nter (+) cells show lower content of cytoplasmic beta-catenin than V3Nter (−) ones (white arrows), beta-catenin localizing to the adherent junctions (blue arrows). Other cells containing both cytoplasmic V3Nter and beta-catenin show membranous beta-catenin staining (yellow arrows). V3Nter (−) cells show cytoplasmic and nuclear, but not membranous beta-catenin staining (gray arrows).

(C) Decreased colony formation in clone HCT116 V3Nter #2 with respect to HCT116 cells stably expressing empty pcDNA 3.1 (VECTOR). After hematoxylin staining, colonies (seen as dark spots) were digitized using a video camera.

FIG. 6. V3Nter reduces in vitro tumor cell proliferation.

Four clones of HCT116 CRC cells were seeded in triplicates in 24-well plates at 12000 cells per well in McCoy's 5A medium containing 10% FCS.

(A) Cells were synchronized in GI phase of the cell cycle in medium without FCS for three days, then stimulated with 10% FCS for 8; 12; 24 or 48 h, then pulsed with 1 μCi 3HThy/ml at 37° C. for 90 min. Total protein was precipitated with 10% trichloroacetic acid and solubilized with 0.3N NaOH/0.1% SDS. Incorporated 3HThy was measured in a scintillation counter (LS6500, Beckman) and normalized to total protein content.

(B) Twenty-four hours after seeding, unsynchronized cells were incubated with 1.2 mM MTT (3-(4,5-dimethylthiazol-2-yl)-2,5-diphenyl-2H tetrazolium bromide) at 37° C. for 2 h, MTT crystals were solubilized with DMSO and optical density (OD) read at 540 nm on a Multiskan plate reader. MTT test shows mitochondrial succinate deshydrogenase activity of living cells.

FIG. 6bis: FZC18 reduces in vitro cell growth of human embryonic kidney (HEK) 293T cells.

HEK 293T cells stably expressing FZC18 (clones #1; #2 and #3) were seeded in 24-well (A and B) or 12-well (C) plates in DMEM containing 10% FCS.

(A) Cells were synchronized in medium without FCS for three days, stimulated with 0% FCS for 24; 48 or 72 h, then pulsed with 1 μCi ^{3H}Thy/ml at 37° C. for 2 h. Total protein was precipitated with 10% trichloroacetic acid, solubilized with 0.3N NaOH/0.1% SDS. Incorporated ^{3H}Thy was measured in a scintillation counter (LS6500, Beckman) and normalized to total protein content.

(B) Time course of cell viability. Cells were incubated with 1.2 mM MTT (3-(4,5-dimethylthiazol-2-yl)-2,5-diphenyl-2H tetrazolium bromide) at 37° C. for 2 h at different time points after seeding. MTT crystals were solubilized with DMSO and optical density (OD) read at 540 nm on a Multiskan plate reader. MTT test shows mitochondrial succinate deshydrogenase activity of living cells.

(C) Time course of cell growth. Cells were counted every day for 8 days using a Malassez cell counter.

FIG. 7. V3Nter reduces in vivo tumor growth of human colorectal carcinoma mouse xenografts.

Three million HCT116 cells (clones VECTOR, V3Nter #1 and V3Nter #2) were subcutaneously injected into both flanks of nu/nu athymic mice (6 mice per group).

(A) V3Nter delays tumor onset on a 30-day time course. The percentage of mice without clinically detectable tumor is shown. HCT116 VECTOR cells elicit tumors in 100% of mice 12 days after injection. By contrast, 80% and 100% of HCT116 V3Nter #1 and #2 mice, respectively, develop tumors 30 days after injection.

(B) V3Nter reduces tumor growth rate on a 22-day time course. Tumor size is measured every other day using calipers. Clone HCT116 V3Nter #2 reduces tumor growth by 10 folds, with respect to clone HCT116 VECTOR.

(C) Twenty days after injection, mice are sacrificed by cervical dislocation and tumors dissected and photographed. Representative images of tumors obtained with the three different clones are shown. Measures are indicated in cm.

FIG. 8. FZC18 binds Wnt3a and suppresses Wnt3a and Wnt1-dependent activation of β-catenin signaling.

(A and B) Wnt3a pulls down V3Nter specifically via the FZC18 domain. EBNA-293 cells were cotransfected with V3Nter (A) or V2Nter (B) and His-tagged mouse Wnt3a. Cell lysates were immunoprecipitated (IP), resolved by 10% PAGE-SDS and immunoblotted with the indicated antibodies. IgG_{H} and IgG_{L} are immunoglobulin heavy and light chains.

(C) A 15-amino acid peptide derived from the CRD of FZC18 (RH3 peptide, SEQ ID NO:9) competes with FZC18 binding to Wnt3a. EBNA-293 cells were cotransfected with mouse V3Nter and with His-tagged mouse Wnt3a. Transfected cells were incubated with 0; 50 or 100 μg/ml of the synthetic peptide RH3 (SEQ ID NO:9) from the CRD domain of FZC18.

Cell lysates were analyzed by immunoblot (10% PAGE-SDS) or coimmunoprecipitated (IP) with monoclonal anti-His antibody.

(D) 3D structure prediction of the FZC18 CRD and modeling of the potential surfaces involved in Wnt-FZC18 interactions. SFRP3 and Frizzled-8 CRD crystal structures were used as templates. The orientation of the CRD surface on the right is rotated 180′ about the vertical axis with respect to left-side images. Blue, N-termini; gray, C-termini; green, surfaces involved in Wnt-CRD interactions inferred from structure-based alignment of FZC18, SFRP3 and FZ8 CRDs and from described mutations affecting Wnt-CRD binding (Dann et al., 2001). Red, localization of the RH3 peptide. Yellow, red and green overlay. 3D structure prediction was done using the Phyre www server and Protein Explorer 2.79.

(E) Reporter gene assays using a β-catenin-TCF responsive reporter (SUPER8XTOPFLASH) or a negative control (SUPER8XFOPFLASH). HEK293T or Huh-7 cells were cotransfected with Wnt3a and the indicated vectors. Results are means of three replicates from a representative experiment. Three independent experiments were performed. Error bars represent standard deviations.

(F) FZC18 and V3Nter inhibit Wnt-1 dependent β-catenin signaling. Reporter gene assays using a β-catenin-TCF responsive reporter (SUPER8XTOPFLASH). HEK293T cells were cotransfected with Wnt1 (black bars) or Wnt3a (white bars) and the indicated vectors. SFRP1, SFRP5, V3Nter and FZC18 inhibit Wnt1- and Wnt3a-dependent activation of β-catenin signaling. By contrast, negative control V2Nter docs not inhibit β-catenin signaling. Results are means of three replicates from a representative experiment. Three independent experiments were performed. Error bars represent standard deviations.

FIG. 9. Modified expression of FCZ18 in fibrosis, cirrhosis and liver cancers

(A) Relative V3 mRNA expression in human liver samples. (B) Small (≦2 cm), well-differentiated HCCs are compared to advanced HCCs. mRNA samples were blotted in triplicates onto nylon membranes and arrays hybridized with ³²P-labeled cDNA under linear-range conditions. Densitometry readings were normalized with an 18S probe. Bar graphs show mean±SD. The Mann Whitney's “U” test was used. NS, non significant; NT, non tumor livers.

FIG. 10. FZC18 is negatively associated with Wnt/beta-catenin pathway activity in vivo. Immunoperoxidase detection (brown) of FZC18, glutamine synthetase (GS) or beta-catenin in normal and tumor livers. Hematoxylin counter-staining (blue).

(A-C) In normal liver, FZC18 (A) is detected around portal tracts (PT). No FZC18 is seen around central veins (CV). GS (B) is detected around CV. (C) Faint cell-membrane beta-catenin.

(D-F) Contiguous sections of tumor liver tissue (TL 325) arising in a cirrhotic nodule. FZC18 (D) is detected in remaining non tumor hepatocytes (NT), compressed by the expansive growth of the tumor, but not in the tumor (T). GS (E) is strong in T and faint in NT. Beta-catenin (F) is detected in cell membranes in NT (thick arrow) and in cytoplasm and nuclei in T (thin arrows) (inset).

(G-L) Contiguous sections of tumor liver tissue (TL 04). Nodule-in-nodule showing faint FZC18 (G), but strong GS (H) staining (asterisks), surrounded by tumor tissue showing strong FZC18, but faint GS staining. (J and K) Higher magnification. (1) Strong FZC18 staining (inset), associated with membranous beta-catenin. (L) Mild FZC18 staining (inset), associated with cytoplasmic and nuclear beta-catenin (arrows).

FIG. 11: Stable HEK293T clones can be used to produce soluble FZC18 A. Schematic structure of V3C18 and FZC18. The C-terminus of the FZC18 module contains a 117 amino-acid frizzled cystein-rich domain (CRD). B. Cell membrane localization of FZC18. The FZC18 module was cloned in frame with a IgK signal peptide in pSecTag2 vector in HEK 293T cells and clones stably expressing the protein were selected. Cells fixed in 4% paraformaldehyde were incubated with rabbit anti-FZC18 and with mouse anti-myc epitope tag (epitopes are shown) followed by incubation with biotin-conjugated goat anti-rabbit, FITC-conjugated goat anti-mouse and streptavidin-conjugated texas red. C. Subcellular localization of FZC18. HEK 293T cell clones stably expressing FZC18 or empty vector were enucleated using a tight-fitting cell douncer, nuclei and debris discarded after centrifugation at 5500 g and supernatants centrifuged at 100 000 g to separate the cytosol and crude membranes, as indicated. FZC18 was detected using anti-myc epitope tag antibody. α-tubulin and caveolin 2 are loading standards. D. FZC18 clones showing different densities of FZC18 (+) cells (brown). Clones were incubated with mouse anti-myc tag antibody followed by anti-mouse peroxidase conjugate. Cells were counterstained with hematoxylin. E and F. Performance of different FZC18 (+) clones to inhibit Wnt3a-induced CRT (β-catenin-T-Cell factor Regulated Transcription). Clones stably expressing FZC18 or empty vector were transiently transfected with a TOPFLASH-luciferase CRT reporter and incubated for 16 hr with conditioned medium from wild-type L cells (MC L) or L cells stably secreting Wnt3a (MC Wnt3a). Results are means of three replicates from a representative experiment. Three independent experiments were performed. Error bars represent standard deviations. F. Immunoblot of FZC18 clones probed with the indicated antibodies (right), after stimulation with Wnt3a (+) or L (−) CM, as indicated. GAPDH is a loading standard. G. Dose-dependent changes in CRT of FZC18-expressing cells in response to increasing concentrations of soluble Wnt3a in the culture medium. Reporter gene assays using a β-catenin-TCF reporter driven by wild-type (TOPFLASH) or a negative control with mutated TCF binding sites (FOPFLASH). Cells stably expressing FZC18 or empty vector were transiently transfected with the CRT reporters and incubated for 16 hr with L or Wnt3a CM. Results are means of three replicates from a representative experiment. Three independent experiments were performed. Error bars represent standard deviations. Twenty μl of medium containing the indicated dilutions of Wnt3a conditioned medium were separated by 10% PAGE-SDS, immunoblotted and probed with anti-Wnt3a antibody. H. Paracrine inhibition of CRT in wild-type HCT116 CRC cells co-cultured with increasing numbers of FZC18-expressing cells. Wild-type HCT116 cells (50 000) were transiently transfected with the CRT reporter (TOPFLASH) or the negative control (FOPFLASH) and co-cultured with increasing numbers of FZC18-expressing cells, as indicated, in the presence of ½ dilution of Wnt3a conditioned medium. To keep constant the total number of cells (250 000), parental HEK293T cells were added to each well. Triplicate wells after TOPFLASH and FOPFLASH luciferase readings were pooled and 20 μg protein immunoblotted for detection of FZC18. GAPDH is a loading standard. Samples are loaded as doublets from TOPFLASH and from FOPFLASH wells, corresponding to the respective histogram bars showing CRT.

FIG. 12: Optimal conditions for producing soluble FZC18 from HEK293T cells Left: HEK293 cells stably expressing FZC18 in suspension culture in serum-free DMEM (Invitrogen). Right: After 72 hr, the cell suspension was harvested, centrifuged at 400 g and the media concentrated by dialysis-lyophilization. Twenty μg of total protein from a reference cell layer preparation (L) containing FZC18 [(+) control] or 200 μg of total protein from conditioned media (M) were resolved by denaturing 7.5% PAGE-SDS electrophoresis, immunoblotted with anti-myc tag antibody and revealed by enhanced chemoluminiscence (Millipore).

FIG. 13: V3Nter is more efficient than SFRP1 for suppressing tumor growth A. V3Nter and V2Nter expression vectors. The dark box is a 47-aa spacer. V5 is an epitope tag. B-D. Female Swiss athymic nude mice (nu/nu, 4-6 weeks old) were injected subcutaneously with 3×10⁶ HCT116 cells into both flanks, as indicated. B. Tumor onset was checked every two days by visual inspection and palpation of the injected area. C. Detectable tumors were measured with calipers and volume was calculated using the formula V=a×b×[(a+b)/2], where a and b are the major and minor axes of the tumor respectively, as described (Lavergne et al., 2003; 2004). D. Mice were sacrificed by cervical dislocation, tumors excised and photographed.

FIG. 14: Combined radiotherapy+FZC18 efficiently suppresses tumor growth in vivo A. HCT116-VECTOR or HCT116-V3Nter cells are subcutaneously injected in nude mice (n=7 per group). Mean tumor volume, as measured with calipers, is shown. Irradiation is performed when tumors measure ˜5 mm in diameter, i.e., day 10 for HCT116-VECTOR (arrow #1) and day 15 (arrow #2) for HCT116-V3Nter cells. By day 23 after injection, some of the mice carrying HCT116-VECTOR tumors show hemorrhagic necrosis of tumors (arrow). B. HCT116 cell tumors on day 23 after injection. HCT116-VECTOR 0 Gy and HCT116-VECTOR 8 Gy arc sacrificed on day 23 after injection because of the necrotic changes observed in the control group. Because of their slow growth rates, which does not provoke tumor necrosis, HCT116-V3Nter 0 Gy and 8 Gy can be kept alive for tumor volume recordings until day 41 after injection.

### EXAMPLE 1

### Material and Methods

Cell Culture, Tissue Samples and mRNA Expression Analysis

Human CRC cell line HCT116 was cultured in McCoy's 5A plus 10% FCS (Invitrogen). Human HCC cell lines HepG2. Huh-6. Huh-7 (de La Coste et al., 1998), and the mouse HCC cell lines mhAT3F and mhAT3FS315 (Vallet et al., 1995) were cultured as described. HEK 293T and 293EBNA cells were cultured in DMEM (Invitrogen), plus 10% FCS. Human tissue samples and mRNA were obtained as described (Musso et al., 2001a), complying with the guidelines of the National Steering Committee of HCC (INSERM, Paris). Relative mRNA expression was assessed using mRNA arrays hybridized with ³²P-labelled cDNAs normalized to 18S under linear-range conditions (Musso et al., 2001a) or by QRT-PCR using SYBR Green PCR Master Mix (Applied Biosystems) and the ABI Prism 7000 (Perkin Elmer). Expression was normalized to 18S and to a calibrator. Primers were designed with Primer 3 on the www. The following primers were used: Forward primer: 5′-GCTICTCTCTCCTCCTTGCTG-3′. (SEQ ID NO: 10) and reverse primer: 5′-GAGAGTCCTTGGCTGTCTGG-3′ (SEQ ID NO: 11).

cDNA Clones

Full-length V2 and V3 C18 cDNAs were described (Elamaa et al., 2003). Human V2Nter and V3Nter cDNAs were PCR-cloned in frame with a V5 tag into pcDNA3.1 (Invitrogen). The open-reading frame for the V3Nter construct is as set forth in SEQ ID NO:12, Mouse V2Nter and V3Nter were cloned into pREP7 (Invitrogen) Human FZC18 was PCR-cloned in pCRII (Invitrogen) using V3Nter as a template, excised with EcoRI and cloned into pSecTag2 (Invitrogen) in frame with an IgK signal sequence and a C-terminal myc tag. The open-reading frame of this construct is as set forth in SEQ ID NO:13. Mouse Wnt1 (pV101, from R. Nusse) and Wnt3a (in pBSII KS+, from J. Kitajewski) cDNAs were transferred to pcDNA 3.1 (Invitrogen) in frame with a C-terminal poly-His tag. SFRP1 and 5 in pcDNA3.1/HisC were from S. Baylin (Suzuki et al., 2004). Super8TOP and Super8FOPFLASH reporters were from R. Moon (Veeman et al., 2003). The Cyclin D1 promoter reporter D1Δ-944pXP2 was from J. Pouyssegur (Lavoie et al., 1996). The normalization Renilla luciferase vector pGL4.70[hRluc] was from Promega. Wild-type beta-catenin and Δ29-48 beta-catenin cDNAs were from R. Grosschedl (Hsu et al., 1998) and Y. Yang (Topo) et al., 2003), respectively. All cDNAs were checked by automatic sequencing (Sequencing Facility, Rennes Hospital, France).

### Reporter Assays

Cells (5×10⁴/well) were transfected on 24-well plates with Lipofectamine Plus (Invitrogen). cDNA was normalized to 250 ng with the appropriate empty vectors. TOP/FOP Flash reporters (15 ng each) or Cyclin D1 promoter reporter (100 ng) were cotransfected with pGL4.70[hRluc] expressing Renilla luciferase. After 48 h, luciferase activity was measured in a scintillation counter (LS6500, Beckman) using the Dual Luciferase Reporter Assay System (Promega).

### Antibodies and Immunological Methods

Anti-C18 antibodies detected human DUF-959, Tsp-1C18 (Saarela et al., 1998), FZC18 (Elamaa et al., 2003) and endostatin (Rehn et al., 2001) as described and mouse DUF-959 (Saarela et al., unpublished). Monoclonal mouse antibodies were directed against: Hsc70, c-myc (9E10) and beta-catenin (E-5) (Santa Cruz); non phosphorylated beta-catenin (8E4 Upstate), myc and V5 tags (Invitrogen), penta-His tag (Qiagen) and glutamine synthetase (BD Biosciences). Polyclonal rabbit anti-cyclin D1 was from Labvision. Secondary antibodies were sheep anti-mouse or goat anti-rabbit coupled to peroxidase (Biorad) or sheep anti-mouse and goat anti-rabbit coupled to FITC or TRITC (Sigma), respectively. Immunoblots and immunohistochemistry were done as described (Musso et al., 2001b). Blot image files were processed with MultiGauge (FujiFilm Lifescience). Microscopes used: Olympus BX60 or confocal Leica TCS NT system on a Leica DMB microscope. Color digital files were prepared with Adobe RGB (1998) on Adobe Photoshop 7.

For immunoprecipitation, mouse His-tagged Wnt3a and mouse V3Nter or V2Nter cDNAs were cotransfected in HEK 293-EBNA cells. Cell lysates were incubated with sheep-anti-mouse-IgG-coated Dynabeads M-280 (Dynal) conjugated with anti-His. Alter washing in RIPA buffer, complexes were eluted in denaturing sample buffer, resolved by 10% PAGE-SDS and immunoblotted with anti-His antibody (to detect Wnt3a) or with anti-DUF-959 (to detect V3Nter or V2Nter). To compete with the Wnt3a-V3Nter interaction, the synthetic peptide RH3 (AWGRFLHTNCHPFLA) from V3Nter CRD was added to culture media.

### Colony Formation Assay and Flow Cytometry

Assays were performed as described (Suzuki et al., 2004). Cells were transfected using Lipofectamine Plus (Invitrogen), stripped and plated in triplicates in 100-mm (colony formation) or 6-well plates (flow cytometry) 24 h after transfection and selected with 0.6 mg ml⁻¹ G148 or 0.5 mg ml⁻¹ zeocin (Invitrogen).

### Tumor Xenografts and Irradiation in Nude Mice:

Female Swiss athymic mice (nu/nu, 4-6 weeks old) were purchased from Iffa Credo Laboratories (L'Arbresle, France), housed under aseptic conditions and cared for in accordance with the guidelines for the Laboratory Animals of INSERM and of the University of Rennes (France). The animal studies and experimental protocols were approved by the local Experimental Animal Platform.

For the xenograft tumor growth assay, HCT116 human colorectal carcinoma cell clones stably transfected with control or V3Nter vectors were cultured in Mc Coy's 5A medium supplemented with 10% FCS at 37° C. in 5% CO₂. After stripping from culture flasks with Trypsin/EDTA (Invitrogen, France) and counting, 3×10⁶ cells were injected subcutaneously in 100 μl of serum-free medium into both flanks of each mouse (n=3 mice/group). The experiments were repeated twice. Tumor onset was checked every two days by palpation of the injection areas. Those animals presenting clinical signs of distress (weight loss, lethargy) and/or showing tumors larger than 1 cm, with clinical signs of tumor necrosis or hemorrhage (bluish surface) were immediately sacrificed. Tumors were measured every two days for 3 weeks with calipers and tumor volume (V) was calculated according to the formula V=a×b×[(a+b)/2], where a and b are major and minor axes of the tumor, respectively. After this follow-up period, mice were sacrificed by cervical dislocation, photographed using a macroscopic photography station equipped with an Olympus 4M pixel digital camera and autopsied. Tumors were dissected out from surrounding tissues, frozen in liquid nitrogen and stored at −80° C. until use. The following macroscopic features were recorded: tumor vascularization, necrosis, adherence to skin/bone/fascias and infiltration of soft tissues. Visual inspection of thoracic and abdominal organs was routinely performed to exclude concurrent pathology. Irradiation was performed in collaboration with F. Paris, Inserm U601, Nantes, France. A Faxitron machine (HP) was used. Settings were 160 kV; 6.3 mA, using a 0.5 mm cupper filter. Before irradiation, mice were administered an intra-peritoneal combination of 65 mg/kg ketamine (Panpharma) and 5 mg/kg xylazine (Bayer) in PBS. Dose delivery was 1.17 Gy/min at a 50 cm focus-object distance. During irradiation, mice were protected using lead screens, allowing localized irradiation of tumors. Mock-irradiated mice underwent the same treatment, including transfer to the irradiation machine under anesthesia, without x-ray delivery. Optimization experiments on 10 mice receiving

VECTOR-HCT116 cells showed that the optimal scheme was a single N Gy administration, blocking tumor growth for 8 days (results not shown).

### Statistics

Differences between means were assessed by the Mann-Whitney's “U” or the Student's “t” tests, as indicated. Bivariate relationships were calculated by the Spearman's rank-order correlation coefficient R or Goodman-Kruskal's Gamma (Statistica 7.1, StatSoft 2006).

### Results

### V3Nter is a Cryptic Inhibitor of Wnt/beta-catenin Signaling.

SFRP1, SFRP2 and SFRP5 suppress beta-catenin—T-cell factor (TCF)-regulated transcription (CRT) from a TCF/LEF responsive reporter in the CRC cell line HCT116 (Suzuki et al., 2004).

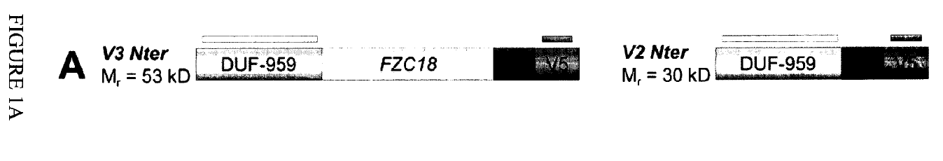

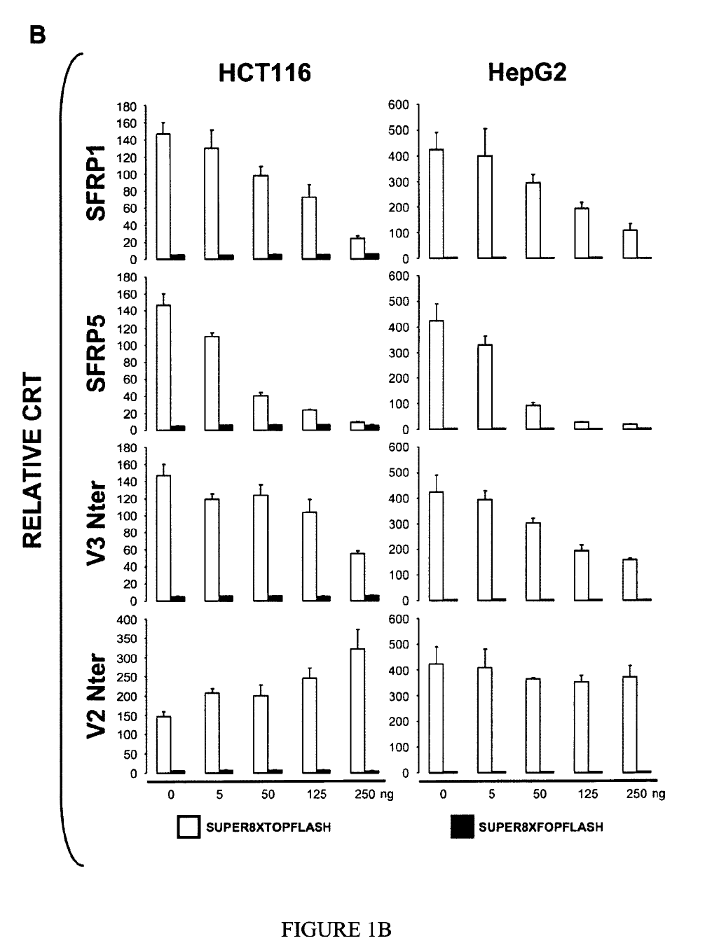

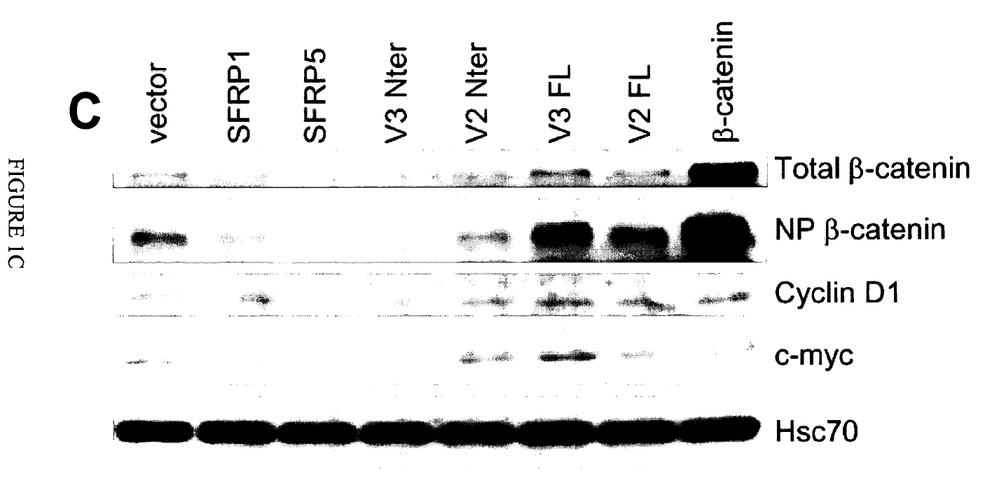

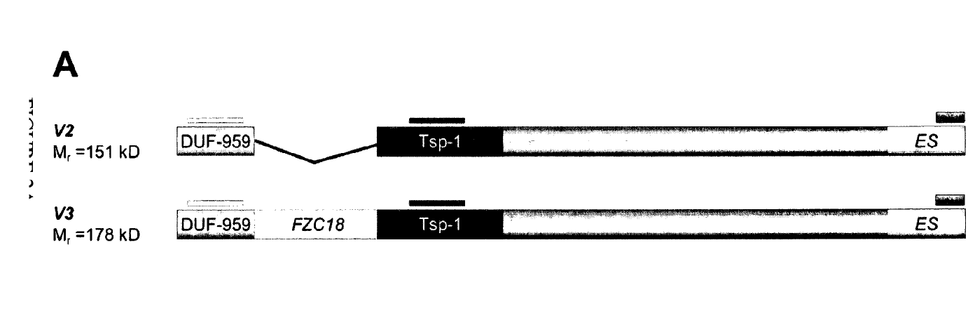

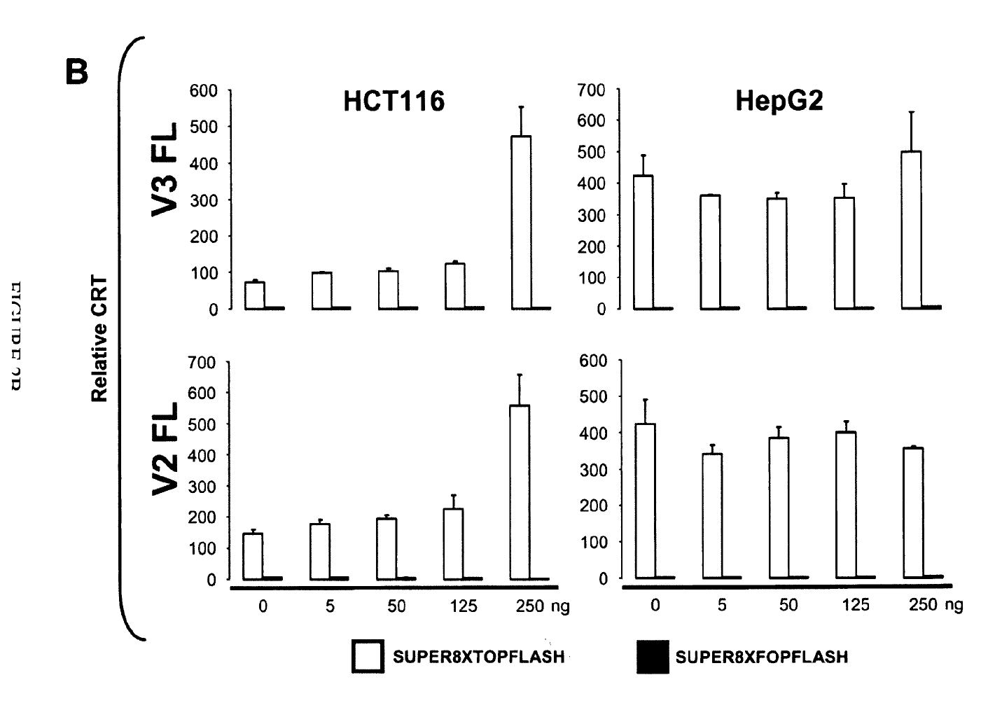

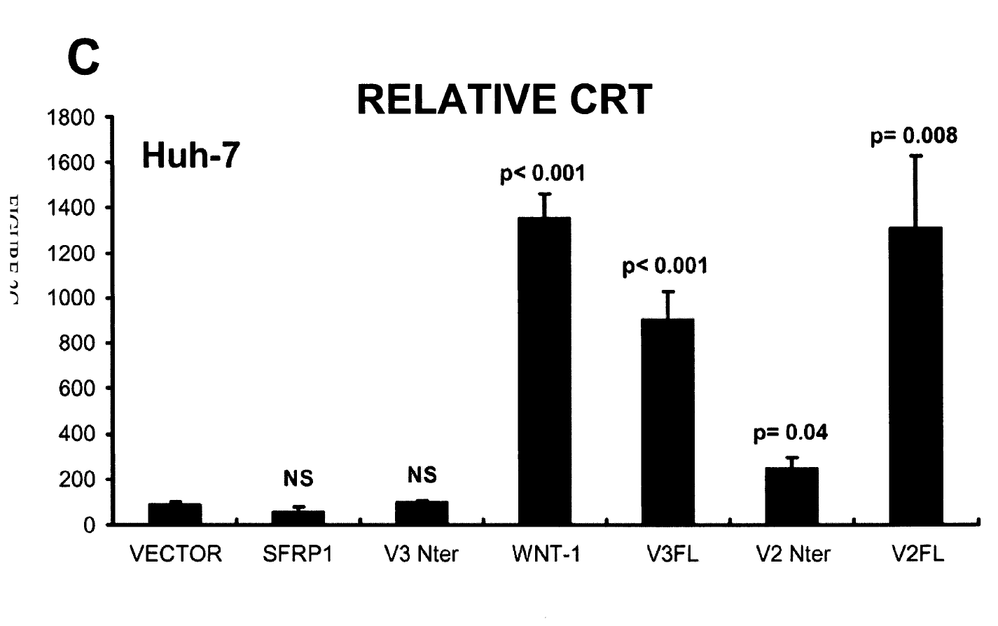

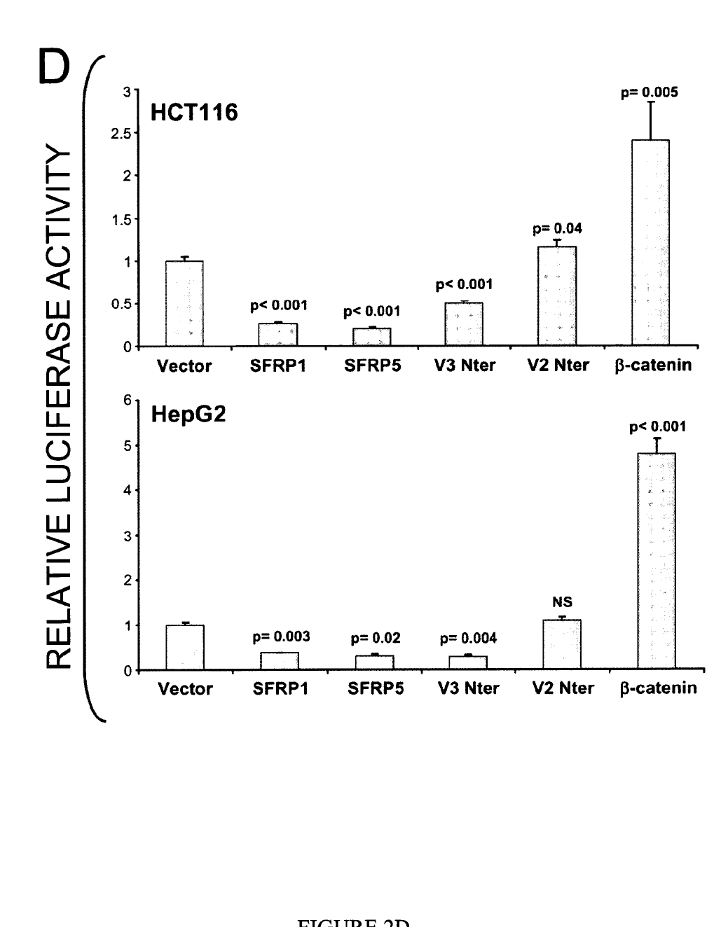

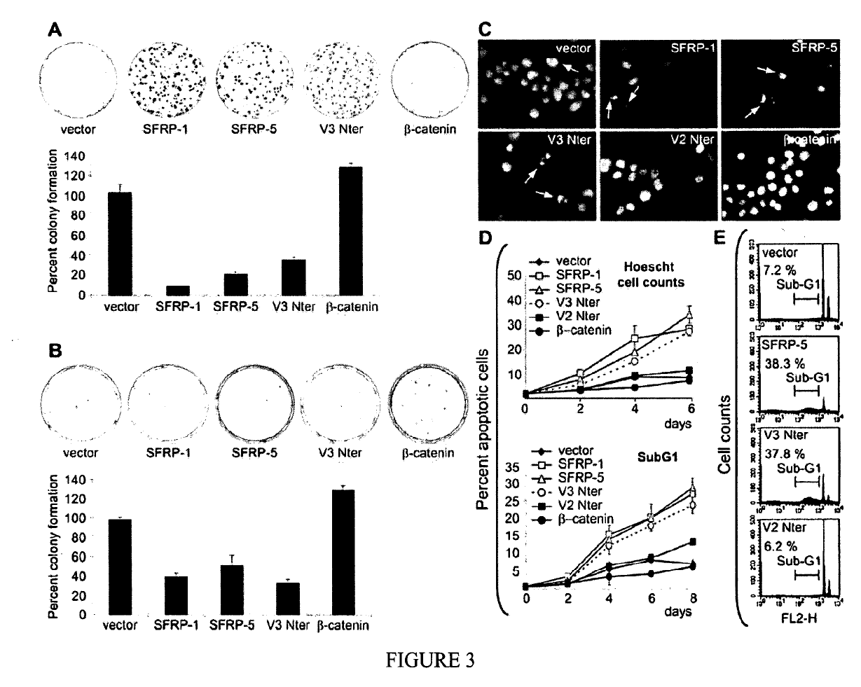

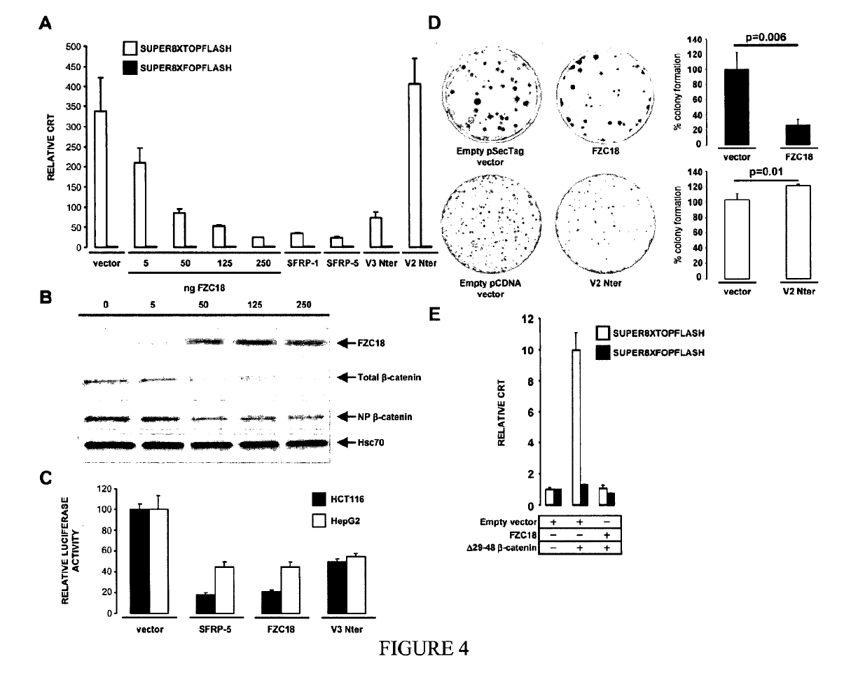

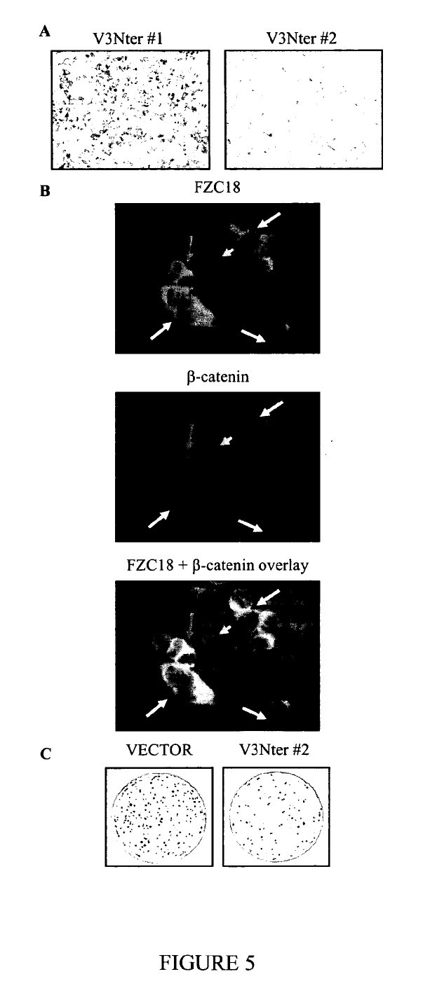

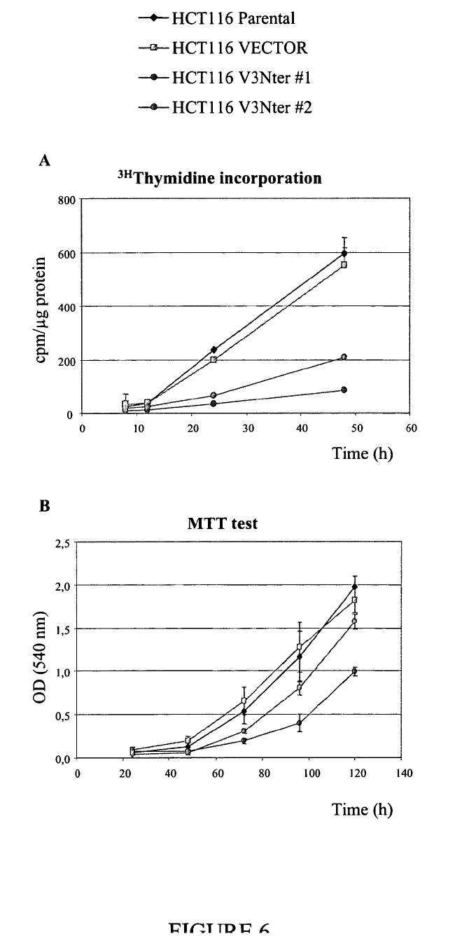

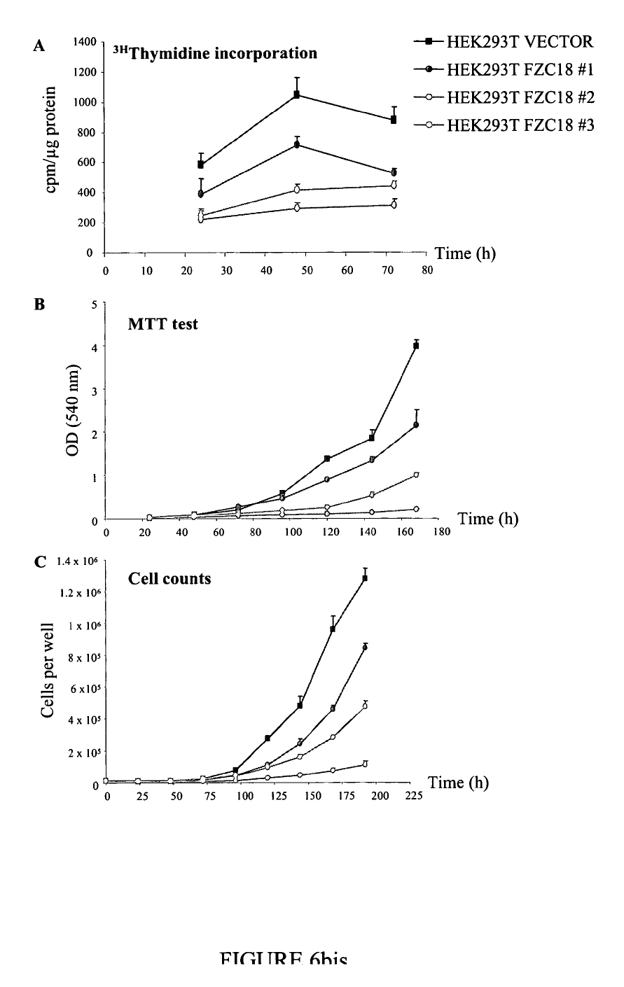

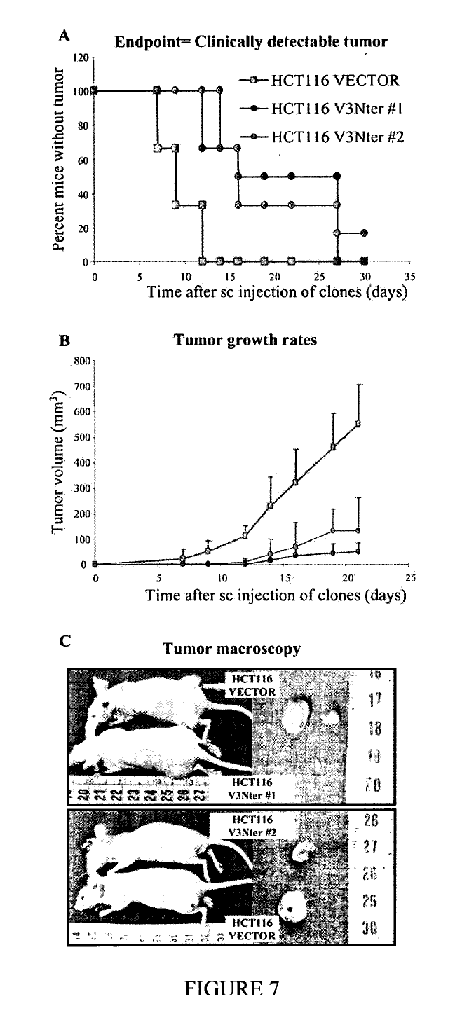

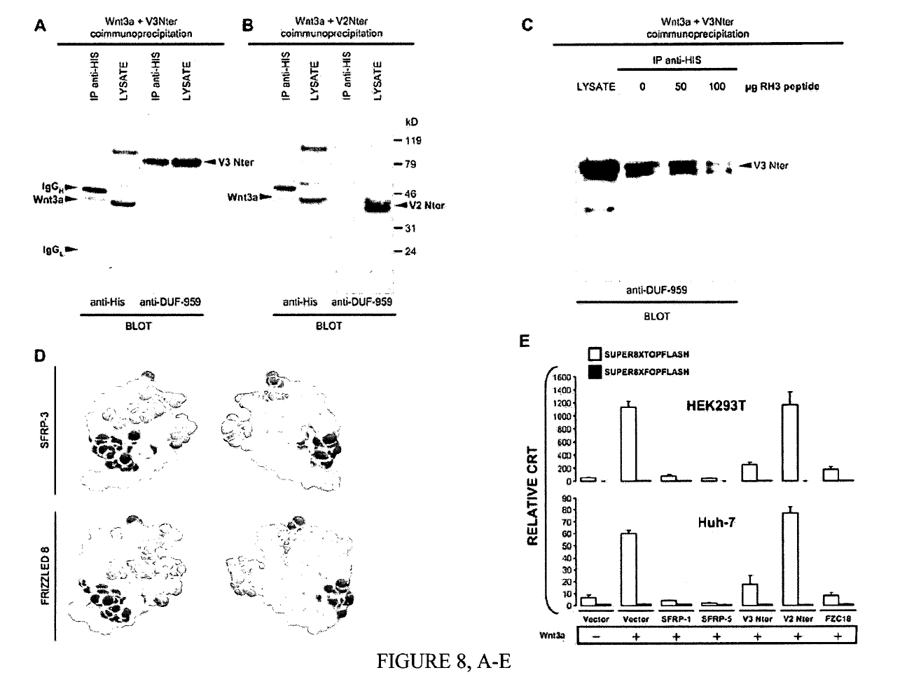

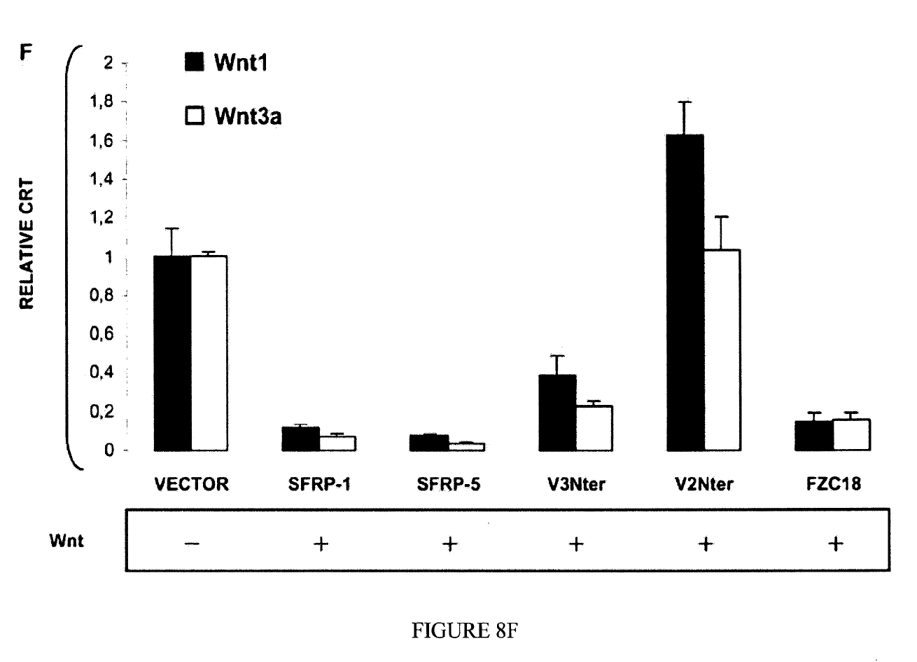

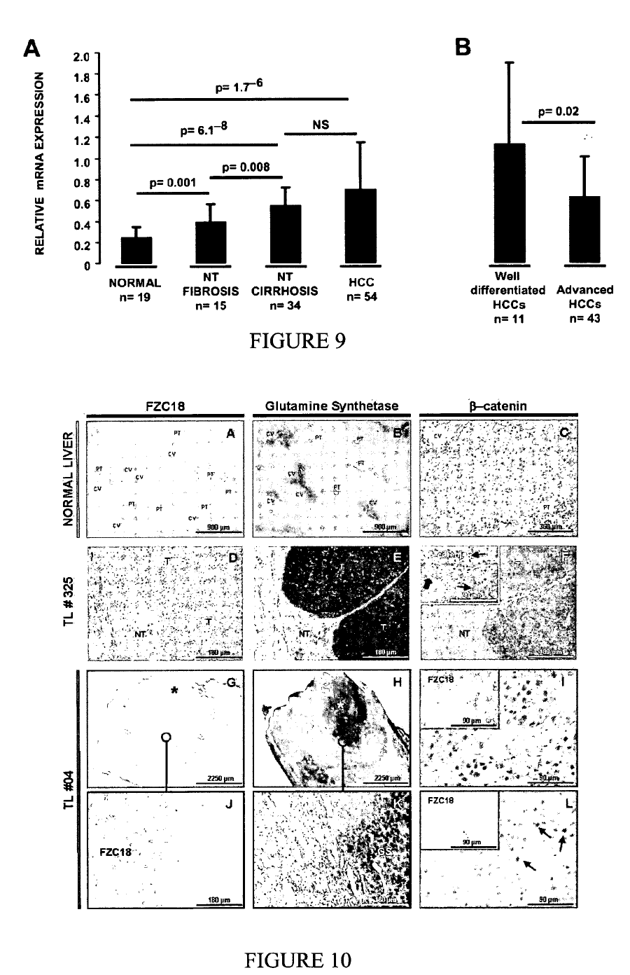

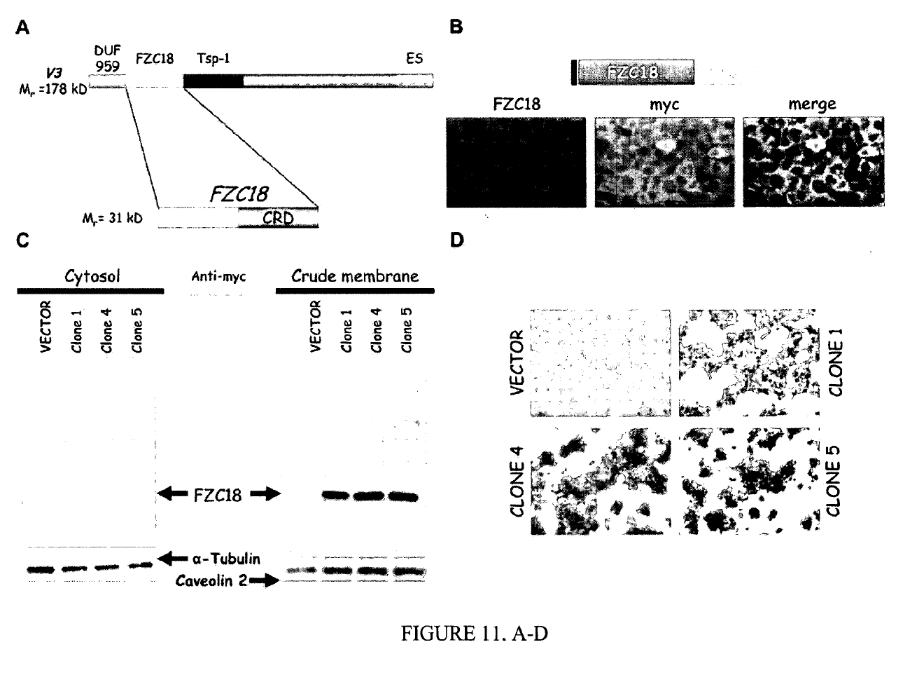

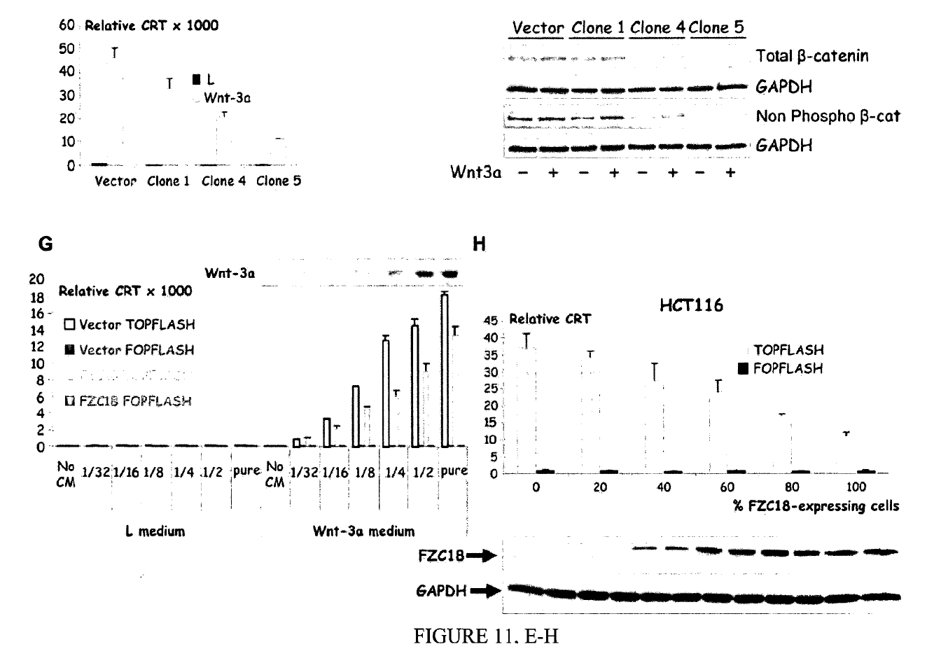

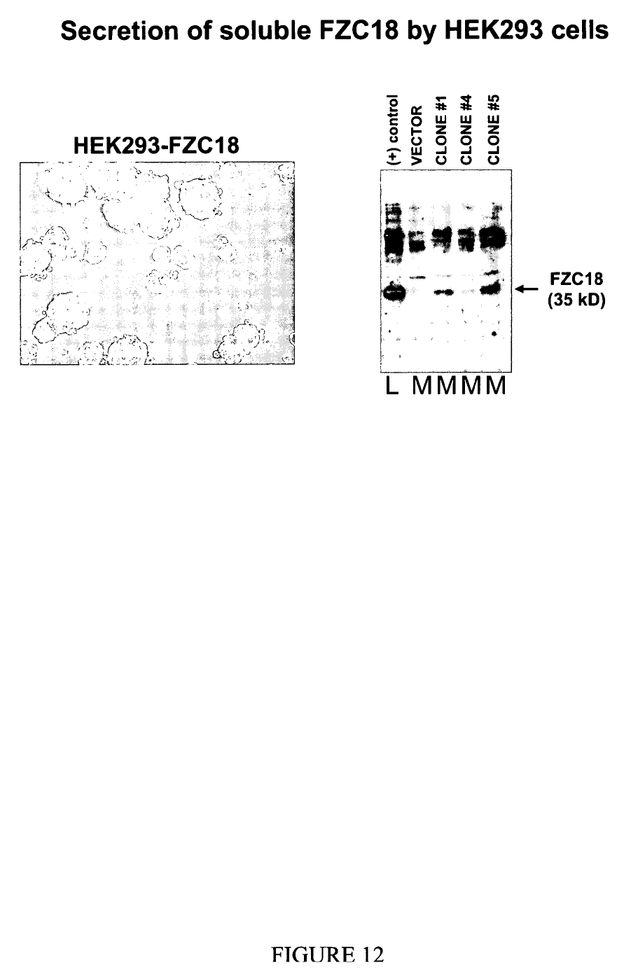

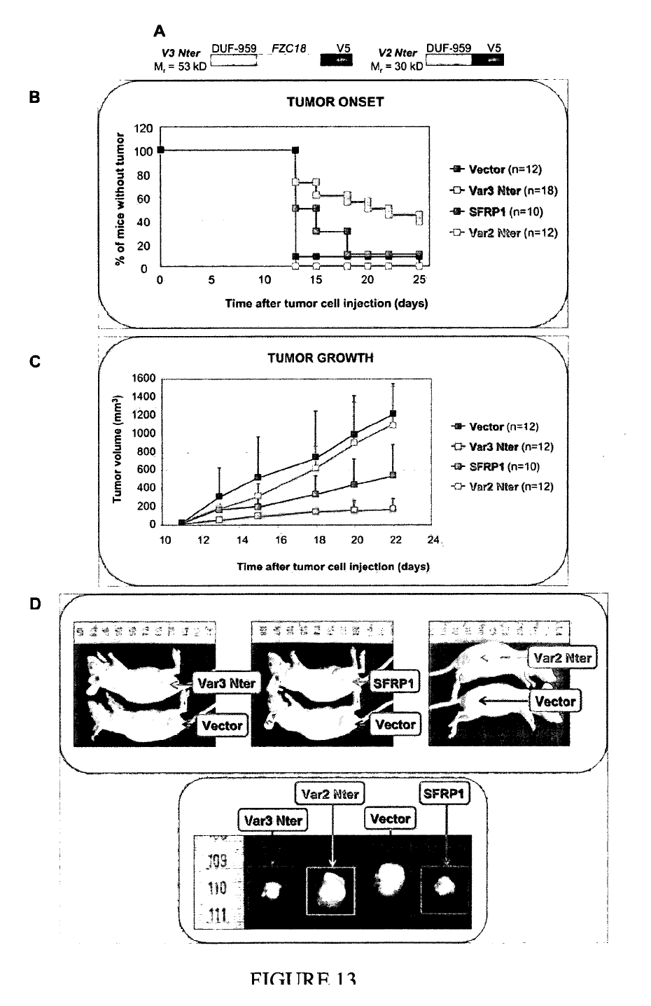

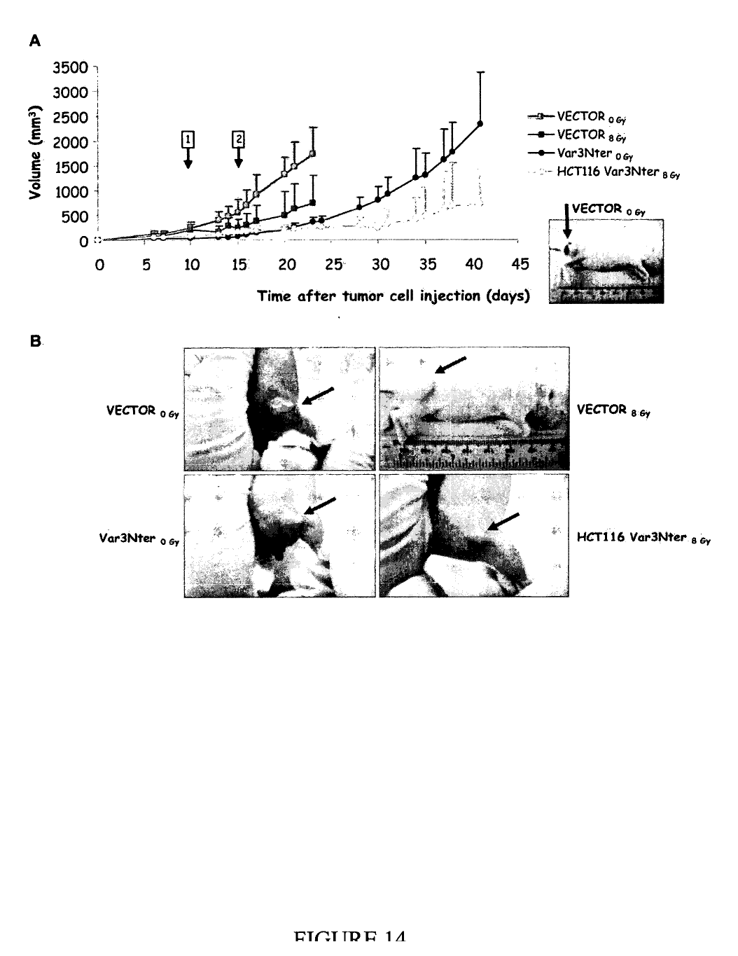

We constructed an expression vector including the natural signal peptide+DUF-959+FZC18 modules and 47 on from the Top-1C18 domain common to all C18 variants that we called V3Nter (FIG. 1A). As a control, V2Nter included the same sequences, but lacked FZC18 (FIG. 1A).

We asked whether V3Nter and V3FL could inhibit Wnt/beta-catenin signaling in cell lines carrying activating beta-catenin mutations, HCT116 (beta-catenin ΔS45) and HepG2 (HCC, beta-catenin Δ25-140) and used SFRP1 and SFRP5 as controls. Ectopic expression of SFRP1, SFRP5 or V3Nter suppressed CRT in a dose-dependent manner (FIG. 1B). By contrast, V2Nter (FIG. 1B), V2FL and V3FL (FIG. 2B), increased CRT in HCT116, but not in HepG2 cells. The increase in CRT by V2Nter, V2FL and V3FL was also observed in other cell lines. In the well-characterized human HCC cell line Huh-7 [wild-type beta-catenin, baseline Wnt/beta-catenin signaling (de La Coste et al., 1998)] Wnt1, V2FL and V3FL increased CRT by more than 10 folds and V2Nter by 2.9 folds (FIG. 2C). Similar results were obtained in the mhAT3F1015 mouse HCC cell line (not shown).

Transient overexpression of V3Nter resulted in a reduced protein content of total beta-catenin, non phosphorylated beta-catenin, c-myc and cyclin D1 in HCT116 cells (FIG. 1C). Consistently with data on CRT, V2FL and V3FL did not reduce the protein content of total and non phosphorylated beta-catenin, cyclin D1 or c-myc (FIG. 1C).

Consistently with decreased cyclin D1 protein expression, reporter gene assays using the cyclin D1 promoter (Lavoie et al., 1996) upstream of luciferase cDNA confirmed that SFRP1, SFRP5 and V3Nter decreased cyclin D1 promoter activity in HCT116 and HepG2 cells (FIG. 2D). Remarkably, overexprcssion of beta-catenin increased cyclin D1 promoter activity by 2.4 and 4.8 folds in HCT116 and HepG2 cell lines, respectively. Taken together, these data show that V3Nter can inhibit beta-catenin signaling in cancer cells carrying activating beta-catenin mutations and that the biological activity of the frizzled CRD is cryptic within full-length cell surface C18.

### V3Nter Inhibits Tumor Cell Growth Through Increased Cell Death.

Based on these findings, we analyzed the effects of V3Nter on growth and death of tumor cells. Decreased colony formation occurred in HCT116 and HepG2 cells overexpressing V3Nter. Inhibition of clonogenesis by V3Nter, SFRP1 and SFRP5 were within the same range in both cell lines (FIG. 3, A and B). beta-catenin induced a ˜30% increase in colony formation in HCT116 and HepG2 cells, in consistency with data on cyclin D1 promoter activity, thus supporting the hypothesis that despite activating beta-catenin mutations, cells can still respond to stimuli inducing further increases in beta-catenin levels. HCT116 cells expressing SFRP1, SFRP5 or V3Nter showed chromatin condensation, nuclear fragmentation, numerous apoptotic bodies and an overall low cell density. By contrast, higher cell densities and rare apoptotic bodies were seen in cells expressing V2Nter, beta-catenin or vector alone (FIG. 3C). Numbers of morphologically apoptotic Hoescht-stained cells and flow cytometry analysis of subG1 cells showed that V3Nter induced tumor cell death within the same range as SFRPs (25 to 35%) in HCT116 cells (FIG. 3D). Similarly, SubG1 analysis on HepG2 cells showed 35-40% cell death (FIG. 3E). As expected, vector alone and V2Nter showed baseline levels of cell death in HCT116 and HepG2 cells (FIGS. 3D and E).

### FZC18 Suppresses Wnt/beta-catenin Signaling.

To test whether FZC18 alone was active, we cloned the FZC18 module in frame with the IgK signal sequence and a C-terminal c-myc tag. Expression in mhAT3F1015 cells revealed a soluble ˜35 kD N-glycosylated polypeptide in cell conditioned medium (data not shown). FZC18 suppressed CRT activity and total and non-phosphorylated beta-catenin stabilization in a dose-dependent manner (FIGS. 4A and B). In addition, FZC18 downregulated cyclin D1 promoter activity (FIG. 4C) and decreased colony formation (FIG. 4D) by 75% compared with cells expressing vector alone. In contrast, V2Nter induced a 21% increase in colony formation with respect to empty vector, consistently with data on CRT and cyclin D1 promoter activity. Additionally, in Huh-7 NEC cells overexpressing a stabilized form of beta-catenin ({29-48 beta-catenin), FZC18 suppressed the increase in CRT (FIG. 4E), confirming that FZC18 can inhibit CRT in cells carrying activating beta-catenin mutations.

### Effect of FZC18 in Stable Human Colorectal Cancer Cell Lines In Vitro.

V3Nter-expressing HCT116 clones were selected with 0.6 mg/ml G418 and screened by immunoblot with anti-V5 epitope tag and anti-FZC18 antibodies. By immunoperoxidase, clones V3Nter #1 and #2 respectively showed intense staining of ˜50% of cells and moderate staining of 100% of cells (FIG. 5A). V3Nter (+) HCT116 cells showed lower cytoplasmic beta-catenin content than V3Nter (−) ones, beta-catenin preferentially localizing to the cell membranes and the adherent junctions (FIG. 5B). V3Nter inhibited in vitro tumor cell growth in colony formation assays (FIG. 5C). Consistently, V3Nter (+) HCT116 cells showed lower rates of DNA synthesis on a 48 h time course (FIG. 6A), resulting in decreased cell growth on a 120 h time course (FIG. 6B).

### Production of FZC18 by Human Embryonic Kidney Cells In Vitro

Different approaches could be undertaken to obtain purified V3Nter or FZC18 proteins. As glycosylation may be important for cell surface targeting and solubility of FZC18, we favored stable expression by mammalian cells, a convenient and widely accepted approach for extracellular matrix and cell surface proteins (Sasaki et al., 1998). We thus produced HEK293T cells stably expressing FZC18. Consistently with HCT116 cells stably expressing V3Nter, FZC18 Clones #1, #2 and #3 showed lower rates of in vitro DNA synthesis (FIG. 6bisA), cell growth (FIG. 6bisB) and cell counts on a 8-day time course (FIG. 6bisC) than cells stably expressing the empty vector. These clones may be used to produce soluble FZC18 in cell conditioned media.

### Effect of FZC18 In-Vivo:

The HCT116 cell lines were further tested on a mouse xenograft tumor model (FIG. 7). On a 30-day time course, V3Nter retarded tumor onset (FIG. 7A). Indeed, 12 days after subcutaneous injection of HCT116 VECTOR cells, 100% of mice developed palpable tumors. By contrast, by 12 days, 60% of mice injected with V3Ntcr clones had no palpable tumor. Respectively, 80% and 100% of HCT116 V3Nter #1 and #2 mice developed palpable tumors 30 days after cell injection. Consistently with in vitro data, V3Nter reduced tumor growth rate by several folds on a 22-day time course in nude mice (FIG. 7, B and C).

FZC18 Binds Wnt3a, Suppressing Wnt3a-Dependent Activation of Beta-Catenin Signaling.

EBNA-293 cells were cotransfected with His-tagged Wnt3a and either mouse V3Nter or V2Nter expression vectors. Wnt3a pulled down V3Nter but not V2Nter (FIG. 8A), as shown by immunoblotting with anti-DUF-959 antibody. In addition, previous incubation of transfected cells with increasing concentrations of a 15-aa peptide named RH3 (SEQ ID NO:9) derived from the CRD of FZC18 competed with FZC18 pull-down by Wnt3a (FIG. 8B), demonstrating that Wnt3a interacts directly with the CRD of FZC18. Then, we searched for in silico models predicting the 3D structure of the FZC18 CRD using threading algorithms that seek for template proteins with well-characterized crystal structures in PDB databases (Phyre www server). Two highly significant matches were mouse SFRP-3 and FZ 8, showing 32% and 22% identity, respectively. E-valucs were 1.4×10⁻¹⁵ for SFRP-3 and 6.6×10⁻¹⁵ for FZ 8, with an estimated precision of 100% for both models. Similar results were obtained by homology modeling using the www server HHpred, indicating 100% probability that the predicted 3D model of FZC18 CRD matches the templates SFRP-3 and FZ 8 (p=0). Next, we looked at the localization of the competing peptide RH3 on the putative surface of the FZC18 CRD. Running the above models on Protein Explorer 2.79 showed that RH3 lies at the FZC18 CRD solvent-exposed surface (FIG. 8C). In addition, the competing peptide lies adjacent to and partially overlaps residues involved in Wnt-mFZ8 CRD interactions (Dann et al., 2001). Consistently, cotransfection of mouse Wnt3a and human FZC18 or V3Nter with a TCF-responsive reporter in the HEK293T and in the Huh-7 cell lines showed that FZC18 and V3Nter suppressed Wnt3a-induced CRT by more than 80%. Similar results were obtained when Wnt1 was cotransfected, indicating that FZC18 and V3Nter also suppress Wnt1-induced CRT (FIG. 8F). Taken together, these data indicate that FZC18 can function as a SFRP-like bioactive polypeptide quenching at least Wnt1 and Wnt3a in the tumor microenvironment. Since the RH3 peptide competes with FZC18 for binding to Wnt3a, it is expected that it should also inhibit Wnt3a-dependent activation of beta-catenin signaling. This is the first demonstration of an extracellular matrix-derived collagen fragment inhibiting two major prototypes of canonical wnt signaling, Wnt1 and Wnt3a.

### Expression of V3 of C18 Liver Tissue

Low expression of the V2 mRNA is associated with tumor progression and reduced disease-free survival in HCCs (Musso et al., 2001a). Although V2 and V3 share a common promoter, V3 is additionally regulated by alternative splicing of FZC18 to produce V2 (Elamaa et al., 2003). Analysis of mRNA arrays from 122 frozen liver samples included normal livers from 19 subjects, 54 HCCs and 49 matching non tumor livers. V3 mRNA levels were higher in fibrotic and cirrhotic livers than in normal livers (FIG. 9A), indicating that the expression of V3 increases during tissue remodeling. Small (≦2 cm) well-differentiated HCCs showed higher V3 mRNA levels than advanced HCCs (FIG. 9B). The mean±SD size of both groups was 1.3+0.38 cm and 6.5+4.6 cm, respectively (p=3×10⁻⁷). In addition, V2 and V3 mRNA levels were positively correlated (R=0.61, n=122, p=1.2×10⁻¹³) As previously shown for V2 of C18 (Musso et al., 2001a), these findings indicate that higher FZC18 mRNA expression is associated with less aggressive tumors.

### Negative Correlation Between FZC18 Expression and Beta-Catenin Pathway Activity In Vivo

Immunoreactivity for FZC18, beta-catenin and glutamine synthetase (GS) was assessed on serial sections of normal, cirrhotic livers and HCCs (FIG. 10). The intensity was semi-quantitatively recorded using a 5-point scale, from absent (−) to strong (++++), by comparing the staining in the tumor with adjacent non-tumor tissue, as described (Zucman-Rossi et al., 2007). In normal liver, FZC18 was periportal (FIG. 10A), contrasting with the well-characterized pericentral vein localization of GS (FIG. 10B) (Benhamouche et al., 2006), suggesting that FZC18 is detected in zones of low beta-catenin pathway activation. In HCCs, mild FZC18 signal was detected at sites where GS was strong and beta-catenin was cytoplasmic or nuclear (FIG. 10, D-F). Conversely, strong FZC18 signal was detected at sites where GS staining was mild and beta-catenin was associated with cell membranes (FIG. 10, G-H). Consistently, statistical analysis of data from 24 tumor nodules indicated that FZC18 was negatively correlated with GS (γ=−0.42; p=0.02; n=24) and cytoplasmic beta-catenin staining (γ=−0.47; p=0.02; n=23). Conversely, FZC18 was positively correlated with cell membrane beta-catenin staining (γ=0.67; p<0.001; n=23). As expected (Zucman-Rossi et al., 2007) nuclear and cytoplasmic beta-catenin were positively correlated (γ=0.89; p<0.001; n=23), as well as GS and nuclear (γ=0.74; p<0.001; n=23) or cytoplasmic beta-catenin (γ=0.63; p<0.001; n=23). These data demonstrate that the FZC18 module is associated with inhibition of Wnt/beta-catenin signaling in the tumor microenvironment.

### EXAMPLE 2

### Soluble FZC18 Can be Produced for Therapeutic Purposes

In example 1, we have shown that owl⁻expression of FZC18 inhibits β-catenin signaling in cells carrying activating β-catenin mutations through autocrine signaling. However, in the physiological setting, tumor cells receive signals from their microenvironment, including cell surface proteins in neighboring cells and soluble factors in the interstitial space. Thus, we prepared clones of FZC18 (+) Human Epithelial Kidney 293T (HEK 293T) cells, a well-characterized cell line capable of secreting glycosylated proteins at high titers (Hsieh et al., 1999), with the aim of confirming that extracellular FZC18 can inhibit β-catenin signaling. Immunostaining with anti-FZC18 and anti-myc tag antibodies confirmed that FZC18 was mainly detected at the cell surface (FIG. 11, A and B). Similar results were obtained after cell fractionation of three FZC18 (+) clones, indicating that FZC18 was only detected in the cell membrane fraction (FIG. 11C). Three clones were tested: clones #1 and #4 were picked from single colonies growing in 100 mm petri dishes. By contrast, clone #5 contained a mixture of colonies. The three clones tested showed different densities of FZC18 (+) cells, as revealed by immunoperoxidase staining (FIG. 11D). These clones did not undergo further limiting dilution cloning to avoid generating subcloning artifacts. HEK293T cell clones stably expressing VECTOR or FZC18 were incubated with a ½ dilution of Wnt3a conditioned medium. FZC18 (+) clones showed an impaired induction of CRT (β-catenin-T-Cell factor Regulated Transcription), as assessed using TOP/FOPFLASH-luciferase reporters (FIG. 11E), and lower total and active (non phosphorylated) β-catenin levels, as observed by immunoblot. Impaired CRT induction was associated with the density of FZC18 (+) cells seen in FIG. 11D. These findings were confirmed in a dose-response assay using clone #5 and increasing concentrations of Wnt3a in the conditioned medium (FIG. 11G).

HCT116 colorectal carcinoma (CRC) cells carry a heterozygous β-catenin mutation (S45 +/−), which blocks β-catenin phosphorylation, thus constitutively activating the Wnt/β-catenin signaling pathway (Chan et al., 2002). We (see Example 1) and others (Suzuki et al., 2004) have previously shown that HCT116 cells can respond to exogenous Wnts by further increasing β-catenin signaling, probably through the wild-type β-catenin allele (Bafico et al., 2004). Thus, we transiently transfected parental HCT116 cells with CRT TOP/FOPFLASH reporters and co-cultured them with increasing numbers of FZC18 (+) HEK293T cells in the presence of a ½ dilution of Wnt3a conditioned medium. Results show that the performance of HCT116 cells to increase CRT in response to Wnt3a is inversely proportional to the number of FZC18 (+) cells in their culture microenvironment (FIG. 11H).

Taken together, these findings indicate that FZC18 is secreted and functional. We tested different approaches to favor accumulation of soluble FZC18 in the cell conditioned medium (i.e., varying concentrations of fetal calf serum, cell density, monolayer versus suspension culture). FZC18 was detected in the cell conditioned medium after seeding cells in serum-free medium, thus promoting the formation of cell aggregates (FIG. 12). These findings demonstrate that, under these conditions, at least part of the protein is correctly folded, secreted and soluble. Suspension culture mammalian cells for production of recombinant proteins is a well-documented approach (Wurm, 2004; Jelkmann, 2007). In this case, the use of bioreactors and/or specific culture media and additives to avoid the formation of aggregates significantly increases the yield of the target protein (Belin et al., 2006).

### EXAMPLE 3

### Use of FZC18, Alone or in Combination with Radiotherapy for Treating Cancer

V3Nter is more efficient than SFRP1 for suppressing tumor growth. V3Nter-, SFRP1-, V2Nter and VECTOR-HCT116 cell clones were injected subcutaneously into athymic nude mice. Mice were housed under sterile conditions and the appearance of tumors was checked every two or three days by visual inspection and palpation of the injection area. Once palpable tumors were detected, they were measured every two or three days using electronic callipers. Tumor incidence and growth were not significantly different in VECTOR-HCT116 or in V2Nter-HT116 tumors (FIG. 13, B and C). By contrast, V3Nter delayed tumor onset. Indeed, more than 90% of the mice injected with VECTOR-HCT116 or V2Nter-HT116 cells developed a solid tumor by day 13 whereas only 60% of the V3Nter-HCT116 cell-injected mice showed palpable tumors on day 25 (FIG. 13A). Moreover, growth of V3Nter-HCT116 tumors was significantly slower than that of the VECTOR-HCT116 or V2Nter-HT116 tumors, their mean volume being 7-fold smaller on day 22 (FIG. 13). SFRP1 delayed tumor onset to a lesser extent (FIG. 13). Thus, 70% of the SFRP1-HCT116 cell-injected mice had a tumor on day 17, and 90% on day 25. On day 22, the reduction of tumor growth by SFRP1 was by 2.2 fold, with respect to VECTOR-HCT116 cells.

Combined radiotherapy+FZC18 efficiently suppresses tumor growth in vivo. HCT116-VECTOR or −V3Nter cells were subcutaneously injected into both flanks in nude mice. Every two or three days, mice were examined and palpable tumors measured with electronic calipers. Tumor volume was calculated from two perpendicular measures (a and b), as described (Lavergne et al., 2003 ; 2004): Tumor volume=a×b×[(a×b)/2].

Mice were injected with V3Nter or control vector clones at day 0 (FIG. 14A). Irradiation was carried out when tumors in each group measured ˜5 mm in diameter, to take into account the delay in tumor growth induced by V3Nter, thus allowing tumor irradiation at ˜200 mm³ mean tumor volume for both groups. Thus. VECTOR tumors were irradiated at day 10, where mean±SD tumor volumes (mm³) were VECTOR 0 Gy=250±111, VECTOR 8 Gy=202±131; p=0.45) and V3Nter tumors at day 15, when V3Nter 0 Gy=76±47 and V3Nter 8 Gy=169±47; p=0.002). Of note, larger tumors where assigned to the irradiated V3Nter group to rule out selection bias.

Irradiation delayed tumor growth in all groups (FIG. 14). Indeed, 13 days after irradiation (23 days after tumor inoculation), mean tumor volume decreased 2.3 folds in VECTOR tumors (p=0.004). Similarly, 15 days after irradiation (day 28 after tumor inoculation), a 3.2-fold decrease was observed in V3Nter tumor volume (p=0.0001). However, 23 days after tumor injection, hemorrhagic necrosis of some of the VECTOR-HCTT16 0 Gy tumors led us to sacrifice all irradiated and non irradiated VECTOR-HCTT16 tumors, keeping alive only 0 and 8 Gy V3Nter-HCT116 tumors (FIG. 14A), because of the different growth kinetics of VECTOR and V3Nter tumors (see above, FIG. 13). Thus, 23 days after tumor injection, mean tumor volume was 3 folds higher in VECTOR 8 Gy than in V3Nter 8 Gy (p=0.04, FIG. 14, A and B), indicating an additive effect of V3Nter and radiation therapy on tumor growth in vivo. V3Nter 8 Gy tumors attain roughly the same maxim volume mm al volume (742 mm³) as VECTOR 8 Gy tumors (792 mm³), with a growth delay of 18 days (FIG. 14A). At the end of the experience (day 41 after tumor inoculation and day 26 after irradiation), V3Nter 8 Gy tumors show a 2.5-fold reduction in tumor volume with respect to V3Nter 0 Gy tumors (p=0.005). Importantly, no additional toxicity was observed in V3Nter 8 Gy mice, without significant weight changes among the 4 groups, the mean weight being 25 g (results not shown). This experiment was performed twice, with similar results.

### References

Belin V, Rousselle P (2006) Production of a recombinantly expressed laminin fragment by HEK293-EBNA cells cultured in suspension in a dialysis-based bioreactor. Protein Expr Purif 48: 43-48.
Bafico A. Liu G, Goldin L, Harris V, Aaronson S A (2004). An autocrine mechanism for constitutive Wnt pathway activation in human cancer cells. Cancer Cell 6, 497-506.
Benhamouche, S., Decaens, T., Godard, C., Chambrey, R., Rickman, D. S., Moinard, C., Vasseur-Cognet, M., Kuo, C. J., Kahn, A., Perret, C., and Colnot, S. (2006). Apc tumor suppressor gene is the “zonation-keeper” of mouse liver. Dev Cell 10, 759-770.
Chan T A, Wang Z, Dang L H, Vogelstein B, Kinzler K W (2002). Targeted inactivation of CTNNB1 reveals unexpected effects of beta-catenin mutation. Proc Natl Acad Sci USA 99, 8265-8270.
Clevers, H. (2006). Wnt/beta-catenin signaling in development and disease. Cell 127, 469-480.
Dann, C. E., Hsieh, J. C., Rattner, A., Sharma, D., Nathans, J., and Leahy, D. J. (2001). Insights into Wnt binding and signalling from the structures of two Frizzled cysteine-rich domains. Nature 412, 86-90.
de La Coste, A., Romagnolo, B., Billuart, P., Renard, C. A., Buendia, M. A., Soubrane, O., Fabre, M., Chelly, J., Beldjord, C., Kahn, A., and Perret, C. (1998). Somatic mutations of the beta-catenin gene are frequent in mouse and human hepatocellular carcinomas. Proc Natl Acad Sci USA 95, 8847-8851.
Elamaa, H., Snellman, A., Rehn, M., Autio-Harmainen, H., and Pihlajaniemi, T. (2003). Characterization of the human type XVIII collagen gene and proteolytic processing and tissue location of the variant containing a frizzled motif Matrix Biol 22, 427-442.
Galli, L. M., Barnes, T., Cheng, T., Acosta, L. Anglade, A., Willert, K., Nusse, R., and Burrus, L. W. (2006). Differential inhibition of Wnt-3a by Sfrp-1, Sfrp-2, and Sfrp-3. Dev Dyn 235, 681-690.
He, X. C., Zhang, J., Tong, W. G., Tawfik, O., Ross, J. Scoville, D. H., Tian, Q., Zeng, X., He, X., Wiedemann, L. M., et al. (2004). BMP signaling inhibits intestinal stem cell self-renewal through suppression of Wnt-beta-catenin signaling. Nat Genet 36,1117-1121.
Hoheisel, J. D. (2006). Microarray technology: beyond transcript profiling and genotype analysis. Nat Rev Genet 7, 200-210.
Hsieh J C, Rattner A, Smallwood P M, Nathans J ( )99). Biochemical characterization of Wnt-frizzled interactions using a soluble, biologically active vertebrate Wnt protein. Proc Natl Acad Sci USA 96, 3546-3551.
Hsu, S. C., Galceran, J., and Grosschedl, R. (1998). Modulation of transcriptional regulation by LEF-1 in response to Wnt-1 signaling and association with beta-catenin. Mol Cell Biol 18, 4807-4818.
Jelkmann W (2007) Recombinant EPO production--points the nephrologist should know. Nephrol Dial Transplant 22: 2749-2753.
Kivirikko, K. I. (1993). Collagens and their abnormalities in a wide spectrum of diseases. Ann Med 25, 113-126.
Laurent-Puig, P., and Zucman-Rossi, J (2006). Genetics of hepatocellular tumors. Oncogene 25, 3778-3786.
Lavergne E, Combadiere B, Bonduelle O, Iga M, Gao J L, et al. (2003). Fractalkine mediates natural killer-dependent antitumor responses in vivo. Cancer Res 63, 7468-7474.
Lavergne E, Combadiere C, Iga M, Boissonnas A, Bonduelle O, et al. (2004). Intratumoral CC chemokine ligand 5 overexpression delays tumor growth and increases tumor cell infiltration. J Immunol 173, 3755-3762.
Lavoie, J. N., L'Allemain, G., Brunet, A., Muller, R., and Pouyssegur, J. (1996). Cyclin D1 expression is regulated positively by the p42/p44MAPK and negatively by the p38/HOGMAPK pathway. J Biol Chem 271, 20608-20616.
Lietard, J., Theret, N., Rehn, M., Musso, O., Dargere, D., Pihlajaniemi, T., and Clement, B. (2000). The promoter of the long variant of collagen XVIII, the precursor of endostatin, contains liver-specific regulatory elements. Hepatology 32, 1377-1385.
Mikels, A. J., and Nusse, R. (2006). Purified Wnt5a protein activates or inhibits beta-catenin-TCF signaling depending on receptor context. PLoS Biol 4, e115.
Moon, R. T., Bowerman, B., Boutros, M., and Perrimon, N. (2002). The promise and perils of Wnt signaling through beta-catenin. Science 296, 1644-1646.
Muragaki, Y., Timmons, S., Griffith, C. M., Oh, S. P., Fadel, B., Quertermous, T., and Olsen, B. R. (1995). Mouse Col18a1 is expressed in a tissue-specific manner as three alternative variants and is localized in basement membrane zones. Proc Natl Acad Sci USA 92, 8763-8767.
Musso, O., Rehn, M., Theret, N., Turlin, B., Bioulac-Sage, P., Lotrian, D., Campion, J.-P., Pihlajaniemi, T., and Clément, B. (2001a). Tumor progression is associated with a significant decrease in the expression of the endostatin precursor collagen XVIII in human hepatocellular carcinomas. Cancer Res 61, 45-49.
Musso, O., Theret, N., Heljasvaara, R., Rehn, M., Turlin, B., Campion, J.-P., Pihlajaniemi, T., and Clément, B. (2001b). Tumor hepatocytes and basement membrane producing cells specifically express two different forms of the endostatin precursor collagen XVIII in human liver cancers. Hepatology 33, 868-876.
O'Reilly, M., Boehm, T., Shing, Y., Fukai, N., Vasios, G., Lane, W. S., Flynn, E., Birkhead, J. R., Olsen, B. R., and Folkman, J. (1997). Endostatin: an endogenous inhibitor of angiogenesis and tumor growth. Cell 88, 277-285.
Rehn, M., Hintikka, E., and Pihlajaniemi, T. (1994). Primary structure of the alpha 1 chain of mouse type XVIII collagen, partial structure of the corresponding gene, and comparison of the alpha 1(XVIII) chain with its homologue, the alpha 1(XV) collagen chain. J Biol Chem 269, 13929-13935.
Rehn, M., Hintikka, E., and Pihlajaniemi, T. (1996). Characterization of the mouse gene for the alpha 1 chain of type XVIII collagen (Col18a1) reveals that the three variant N-terminal polypeptide forms are transcribed from two widely separated promoters. Genomics 32, 436-446.
Rehn, M., and Pihlajaniemi, T. (1994). Alpha 1(XVIII), a collagen chain with frequent interruptions in the collagenous sequence, a distinct tissue distribution, and homology with type XV collagen. Proc Natl Acad Sci USA 91, 4234-4238.
Rehn, M., Veikkola, T., Kukk-Valdre, E., Nakamura, H., Ilmonen, M., Lombardo, C., Pihlajaniemi, T., Alitalo, K., and Vuori, K. (2001). Interaction of endostatin with integrins implicated in angiogenesis. Proc Natl Acad Sci USA 98, 1024-1029.
Reya, T., and Clevers, H. (2005). Wnt signalling in stem cells and cancer. Nature 434, 843-850.
Saarela, J., Rehn, M., Oikarinen, A., Autio-Harmainen, H., and Pihlajaniemi, T. (1998). The short and long forms of type XVIII collagen show clear tissue specificities in their expression and location in basement membrane zones in humans. Am J Pathol 153, 611-626.
Sasaki, T., Fukai, N., Mann, K., Gohring, W., Olsen, B. R., and Timpl, R. (1998). Structure, function and tissue forms of the C-terminal globular domain of collagen XVIII containing the angiogenesis inhibitor endostatin. Embo J 17, 4249-4256.
Shih, Y. L., Shyu, R. Y., Hsieh, C. B., Lai, H. C., Liu, K. Y., Chu, T. Y., and Lin, Y. W. (2006). Promoter methylation of the secreted frizzled-related protein 1 gene SFRP1 is frequent in hepatocellular carcinoma. Cancer 107, 579-590.
Suzuki, H., Watkins, D. N., Jair, K. W., Schuebel, K. E., Markowitz, S. D., Chen, W. D., Pretlow, T. P., Yang, B., Akiyama, Y., Van Engeland, M., et al. (2004). Epigenetic inactivation of SFRP genes allows constitutive WNT signaling in colorectal cancer. Nat Genet 36, 417-422.
Tao, Q., Yokota, C., Puck, H., Kofron, M., Birsoy, B., Yan, D., Asashima, M., Wylie, C. C., Lin, X., and Heasman, J. (2005). Maternal wnt11 activates the canonical wnt signaling pathway required for axis formation in Xenopus embryos. Cell 120, 857-871.
Topol, L., Jiang, X., Choi, H., Garrett-Beal, L., Carolan, P. J., and Yang, Y. (2003). Wnt-5a inhibits the canonical Wnt pathway by promoting GSK-3-independent beta-catenin degradation. J Cell Biol 162, 899-908.
Vallet, V., Antoine, B., Chafey, P., Vandewalle, A., and Kahn, A. (1995). Overproduction of a truncated hepatocyte nuclear factor 3 protein inhibits expression of liver-specific genes in hepatoma cells. Mol Cell Biol 15, 5453-5460.
Veeman, M. T., Slusarski, D. C., Kaykas, A., Louie, S. H., and Moon, R. T. (2003). Zebrafish prickle, a modulator of noncanonical Wnt/Fz signaling, regulates gastrulation movements. Curr Biol 13, 680-685.
Wawrzak, D., Metioui, M., Willems, E., Hendrickx, M., de Genst, E., and Leyns, L. (2007). Wnt3a binds to several sFRPs in the nanomolar range. Biochem Biophys Res Commun 357, 1119-1123.
Wodarz, A., and Nusse, R. (1998). Mechanisms of Wnt signaling in development. Annu Rev Cell Dev Biol 14, 59-88.
Wurm F M (2004) Production of recombinant protein therapeutics in cultivated mammalian cells. Nat Biotechnol 22: 1393-1398.
Xu, Y. K., and Nusse, R. (1998). The Frizzled CRD domain proteins including several receptor tyrosine kinases. Curr Biol 8, R405-406.

1. A polypeptide comprising at least 13 consecutive amino acids selected from the amino acid sequence as set forth in SEQ ID NO: 1 or a variant thereof comprising at least 70% identity over said 13 consecutive amino acids, wherein said polypeptide or variant thereof binds to Wnt3a.

2. A polypeptide or variant thereof according to claim 1, wherein said polypeptide comprises at most 600 amino acids.

3. A polypeptide or variant thereof according to claim 1, wherein said 13 consecutive amino acids are comprised in the amino acid sequence as set forth in SEQ ID NO:8.

4. A polypeptide according to claim 1, wherein said polypeptide has the amino acid sequence as set forth in SEQ ID NO:8.

5. A polypeptide according to claim 1, wherein said variant has the amino acid sequence as set forth in SEQ ID NO:9.

6. A polypeptide according to claim 1, wherein said variant is as set forth in SEQ ID NO:3.

7. A nucleic acid comprising a nucleic acid sequence encoding a polypeptide or variant thereof according to claim 1 in frame with a nucleic acid sequence encoding a signal peptide, wherein said nucleic acid sequence encoding a signal peptide is upstream from said nucleic acid sequence encoding a polypeptide or variant thereof according to claim 1.

8. A cell line stably expressing a polypeptide according to claim 1.

9. A method for the treatment of a disease associated-with increased Wnt/beta-catenin pathway activity comprising administering to a subject in need thereof a therapeutically effective amount of a polypeptide or a variant thereof according to claim 1.

10. The method according to claim 9 wherein said disease associated with increased Wnt/beta-catenin pathway activity is selected from the group consisting of colorectal cancers, hepatocellular carcinomas, childhood hepatoblastomas, melanoma, multiple myeloma, lymphoproliferative malignant diseases, breast cancers, desmoids tumors, gastric cancers, Wilms kidney tumors, medulloblastomas, ovarian endometrioid carcinomas, endometrial carcinomas, pancreatic carcinomas, prostate and thyroid carcinomas.

11. The method according to claim 10 wherein said disease associated with increased Wnt/beta-catenin pathway activity is selected from the group consisting of colorectal cancers and hepatocellular carcinomas.

12. The method according to claim 9, wherein said polypeptide or variant thereof or nucleic acid is used in combination with radiotherapy.

13. A method for diagnosing a disease associated with fibrogenesis in a subject, wherein the expression of the variant 3 of collagen 18 is measured in a biological sample obtained from said subject.

14. A method for diagnosing a disease according to claim 13, wherein said disease is a liver disease.

15. A method for diagnosing a disease according to claim 14, wherein said liver disease is liver fibrosis or liver cirrhosis or hepatocellular carcinoma.

16. A method for assessing the severity and/or predicting the outcome of a disease selected from the group consisting of colorectal cancers, hepatocellular carcinomas, childhood hepatoblastomas, melanoma, multiple myeloma, lymphoproliferative malignant diseases, breast cancers, desmoids tumors, gastric cancers, Wilms kidney tumors, medulloblastomas, ovarian endometrioid carcinomas, endometrial carcinomas, pancreatic carcinomas, prostate and thyroid carcinomas, wherein the expression of the variant 3 of collagen 18 is measured in a biological sample obtained from said subject.

17. A method for assessing the severity and/or predicting the outcome of a disease according to claim 16, wherein said disease is selected from the group consisting of colorectal cancers and hepatocellular carcinomas.

18. A method of treating a disease associated with increased Wnt/beta-catenin pathway activity comprising administering to a subject in need thereof a therapeutically effective amount of a nucleic acid of claim 7 in an amount effective to deliver a therapeutically effective amount of a polypeptide encoded by said nucleic acid.

19. A method of treating a disease associated with increased Wnt/beta-catenin pathway activity comprising administering to a subject in need thereof a therapeutically effective amount of a cell of claim 8.
